(19) United States
(12) Patent Application Publication
(10) Pub. No.: US 2015/0241456 A1
(43) Pub. Date: Aug. 27, 2015

(54) METHODS FOR USING LIPID PARTICLES

(71) Applicants: Manas K. HALDAR, Fargo, ND (US); NDSU RESEARCH FOUNDATION, Fargo, ND (US)

(72) Inventors: Erin Nyren-Erickson, Christine, ND (US); Sanku Mallik, West Fargo, ND (US); D.K. Srivastava, Reiles Acres, ND (US); Manas K. Haldar, Fargo, ND (US)

(21) Appl. No.: 14/429,908

(22) PCT Filed: Sep. 19, 2013
(86) PCT No.: PCT/US13/60685

§ 371 Date: Mar. 20, 2015

Related U.S. Application Data
(60) Provisional application No. 61/703,343, filed on Sep. 20, 2012.

(51) Int. Cl. G01N 33/92; C07H 21/00

(52) U.S. Cl. CPC G01N 33/92; C07H 21/00; G01N 2400/40

(57) ABSTRACT

Provided herein are methods for using lipid particles. In one embodiment, lipid particles are used for determining whether a composition includes a charged contaminant. The method includes combining a test composition that includes an analyte with lipid particles to form a mixture, and determining whether there is any change in zeta potential of the mixture and/or average aggregate diameter of liposome aggregates in the mixture. In one embodiment, lipid particles are used for enriching an analyte. The method includes combining a test composition that includes an analyte with lipid particles and multivalent cations to form a mixture, incubating the mixture under conditions suitable for forming a complex that includes the analyte bound to the lipid particle, and separating the complex from the contaminant present in the mixture.

### CROSS-REFERENCE TO RELATED APPLICATIONS

This application claims the benefit of U.S. Provisional Application Ser. No. 61/703,343, filed Sep. 20, 2012, which is incorporated by reference herein.

### GOVERNMENT FUNDING

The present invention was made with government support under CA113746 and CA132034 awarded by the National Institute of Health and under DMR 1005011 awarded by the National Science Foundation. The government has certain rights in the invention.

### BACKGROUND

Glycosaminoglycans (GAGs) are linear polysaccharides composed of disaccharide units of an amino sugar and uronic acid (Zhang et al., 2009, The Handbook of Glycomics. Elsevier: London, UK, 2009). When incubated with phosphatidylcholine liposomes and divalent cations, GAGs cause the aggregation of liposomes (Krumbiegel et al., 1990, Chemistry and Physics of Lipids 1990, 54, 1-7; Satoh et al., FEBS Letters 2000, 477, 249-252). The interaction between liposome charge and GAG concentration to cause this effect has been well documented (Krumbiegel et al., 1990, Chemistry and Physics of Lipids 1990, 54, 1-7; Satoh et al., FEBS Letters 2000, 477, 249-252; Zschornig et al., Colloid Polymer Science 2000, 278, 637-646; Kim et al., J Biol Chem 1977, 252 (4), 1243-9). However, studies conducted to date have focused primarily on mechanism of GAG binding.

Heparin is a naturally occurring GAG which, when fully sulfated, has three sulfate groups per repeating disaccharide unit, making it the most negatively charged naturally occurring polyelectrolyte in mammalian tissues (Voet and Voet, Biochemistry. 3rd ed.; John Wiley & Sons, Inc.: Hoboken, N.J., 2004). Its primary physiological function is highly varied; however its pharmaceutical form (which is typically purified either from porcine intestine or bovine lung) is widely utilized as a drug for the prevention of blood clots in surgery patients (Linhardt et al., Journal of Medicinal Chemistry 2003, 46, 2551-2564).

In 2007-2008, several batches of heparin were found to be contaminated with over-sulfated chondroitin sulfate (OSCS), a product prepared by the synthetic oversulfation of chondroitin sulfate (Maruyama et al., Carbohydrate Research 1998, 306, 35-43) at levels 0.5% by weight to 28% by weight (Beyer et al., Eur J Pharm Sci 2010, 40 (4), 297-304). Over-sulfated chondroitin sulfate has similar but considerably reduced physiological effects as compared to heparin; the anticoagulant effect of oversulfated chondroitin sulfate is approximately 20-25% of that is given by heparin (Satoh et al., FEBS Letters 2000, 477, 249-252). In addition, its intravenous administration was associated with numerous allergic reactions, including 149 deaths (Pan et al., Nature Biotechnology 2010, 28 (3), 203-207). The adverse effects of oversulfated chondroitin sulfate result from a potent anaphylactic response caused by the activation of the kinin-kallikrein pathway, leading to the release of bradykinin (Li et al., Biochemical Pharmacology 2009, 78, 292-300). Other over-sulfated GAGs have also been shown to modulate this response (Pan et al., Journal of Biological Chemistry 2010, 285 (30), 22966-22974).

To circumvent the onset of above noted side effects, many techniques have been explored/developed for the detection of over-sulfated GAG contaminants in commercial preparations of heparin. These include ¹H NMR spectroscopy (Zhang et al., Journal of Pharmaceutical Sciences 2009, 98, 4017-4026), potentiometric strip tests (Kang et al., Analytical Chemistry 2011, 83, 3957-3962), enzyme immunoassay (ELISA) (Bairstow et al., Analytical Chemistry 2009, 288, 317-321), polyanionic sensors (Wang et al., Analytical Chemistry 2008, 80, 9845-9847), colorimetric assays (Sommers et al., Analytical Chemistry 2011, 8, 3422-3420), and activated partial thromboplastin times (aPTT) and prothrombin times (PT) performed with sheep and human plasma Alban et al., Anal Bioanal Chem 2011, 399 (2), 605-20 ( ). While each of these techniques presents advantages, all require specialized equipment, highly-trained personnel, and/or considerable time to obtain results.

### SUMMARY OF THE APPLICATION

In the pursuit of developing an easily adaptable and sensitive protocol for detection of oversulfated GAG contaminates in heparin preparations, we investigated liposome aggregation in the presence of GAG and Mg²⁺, varying both liposome diameter and composition, as well as GAG species and concentration. We developed an assay that is sensitive to the presence of over-sulfated GAG contaminates in heparin preparations, as well as other charged contaminants in other preparations, by measuring changes in aggregate diameter and/or zeta potential of lipid particles.

Provided herein are methods for determining whether a composition includes a charged contaminant. In one embodiment, the method includes combining a test composition with lipid particles to form a mixture, wherein the test composition includes an analyte, and determining the zeta potential of the mixture, determining the average aggregate diameter of liposome aggregates in the mixture, or determining both the zeta potential and the average aggregate diameter of liposome aggregates. When determining the zeta potential, the zeta potential of the mixture is compared to the zeta potential of a control mixture that includes the lipid particles and a reference composition that includes the analyte of known purity. The detection of a difference between the zeta potential of the mixture and the zeta potential of the control mixture indicates the presence of the charged contaminant in the test composition. When determining the average aggregate diameter of liposome aggregates, the average aggregate diameter of liposome aggregates in the mixture is compared to the average aggregate diameter of a control mixture that includes the lipid particles and a reference composition that includes the analyte of known purity. The detection of a difference between the average aggregate diameter of the mixture and the average aggregate diameter of the control mixture indicates the presence of the charged contaminant in the test composition.

In one embodiment, the analyte may include a polymer. In one embodiment, the polymer may include a polynucleotide. In one embodiment, when the polynucleotide is a supercoiled DNA, the charged contaminant includes a relaxed polynucleotide. In one embodiment, the lipid particles include amphipathic molecules having a positively charged hydrophilic region.

In one embodiment, the polymer may include heparin, and in one embodiment the charged contaminant may include glycosaminoglycans (GAGs) that are over-sulfated (such as, for example, dermatan sulfate, chondroitin sulfate, and the combination thereof), under-sulfated, or both over-sulfated and under-sulfated. In one embodiment, the lipid particles may include amphipathic molecules having a zwitterionic hydrophilic region. In one embodiment, at least one amphipathic molecule includes at least one hydrophobic chain that is unsaturated, and in one embodiment, is present in the lipid particle at a concentration of at least 99 mol %. The method may further include digesting the heparin with nitrous acid or with heparinase before the combining.

In one embodiment, the polymer may include a polypeptide. In one embodiment, the analyte may include an organic molecule.

In one embodiment, the zeta potential of the mixture is decreased by at least 5% compared to the control mixture. In one embodiment, the average aggregate diameter of liposome aggregates in the mixture is decreased by at least 5% compared to the control mixture.

In one embodiment, the level of contaminant is at least 0.3% weight of charged contaminant/weight of analyte.

In one embodiment, the lipid particles include liposomes. In one embodiment, the lipid particles include amphipathic molecules having a zwitterionic hydrophilic region. In one embodiment, the mixture further includes a multivalent cation. In one embodiment, the multivalent cation is a divalent cation, such as Mg++. In one embodiment, the multivalent cation is a trivalent cation.

Also provided herein are methods for enriching an analyte. In one embodiment, the method includes combining a test composition with lipid particles and multivalent cations to form a mixture, wherein the test composition includes an analyte and a contaminant, incubating the mixture under conditions suitable for forming a complex that includes the analyte bound to the lipid particle, and separating the complex from the contaminant. In one embodiment, the method further includes exposing the complex to conditions suitable for separating the complex into the analyte and the lipid particle. In one embodiment, the multivalent cation includes a divalent cation. In one embodiment, the analyte is a polynucleotide, such as DNA, RNA, or a combination thereof, and the contaminant includes a polymer, such as LPS, colanic acid, or a combination thereof.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Conditions that are “suitable” for an event to occur, or “suitable” conditions are conditions that do not prevent such events from occurring. Thus, these conditions pennit, enhance, facilitate, and/or are conducive to the event.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Structures of rhodamine (A) and pyranine (B) lipids.

FIG. 2. Average aggregate diameters of DSPC liposome aggregates (A) and POPC liposomes (B); and average zeta potentials of DSPC liposome aggregates (C) and POPC liposomes (D) in the presence of increasing concentrations of heparin (squares), over-sulfated chondroitin sulfate (triangles), over-sulfated dermatan sulfate (circles), and over-sulfated heparin (upside-down triangles).

FIG. 3. TEM images of POPC liposomes with Mg²⁺ only (A): red arrows denote individual liposomes), and aggregated in the presence of heparin (B), over-sulfated chondroitin sulfate (C), over-sulfated dermatan sulfate (D), and over-sulfated heparin (E) magnified 5,000×. Notable is the increase in average size of the aggregates of over-sulfated GAGs over heparin, as well as the polydispersity of these aggregates. Shown also is an image of liposomes aggregated with over-sulfated chondroitin sulfate magnified 25,000× (F). Clearly shown are the clustered bilayers in one section of the aggregate, denoted by the arrows.

FIG. 4. TEM images of DSPC liposomes with Mg²⁺ only (A), and aggregated in the presence of heparin (B), over-sulfated chondroitin sulfate (C), over-sulfated dermatan sulfate (D), and over-sulfated heparin (E) magnified 5,000×. Notable is the polydispersity of these aggregates. Shown also is an image of liposomes aggregated with over-sulfated chondroitin sulfate magnified 25,000× (F). Visible are the closely associated liposomes within a single aggregate.

FIG. 5. DSC traces of DSPC liposomes with heparin (A), over-sulfated chondroitin sulfate (B), over-sulfated dermatan sulfate (C), and over-sulfated heparin (D): liposomes only (T-1), GAG at 1 μM (T-2), GAG at 250 μM (T-3).

FIG. 6. Percent changes for 50 nm diameter liposomes (A, B), 200 nm liposomes (C, D), and 500 nm liposomes (E, F). Shown are percent changes in aggregate diameter (A, C, E) and percent changes in aggregate zeta potential (B, D, F). Concentrations used for this study are 50 nM (squares), 170 nM (circles), and 500 nM (triangles).

FIG. 7. Zeta potentials of liposomal aggregates formed in the presence of heparin contaminated at varying levels with OSCS following digestion using method ‘D’.

### DETAILED DESCRIPTION OF ILLUSTRATIVE EMBODIMENTS

Provided herein are methods for determining whether a composition that includes an analyte also includes a charged contaminant. In one embodiment, the method includes combining a test composition with lipid particles to form a mixture, wherein the test composition includes an analyte, and determining whether a charged contaminant is present in the test composition. The process of determining whether a charged contaminant is present includes determining the zeta potential of the mixture, and/or determining the average aggregate diameter of liposome aggregates in the mixture. Without intending to be limited by theory, it is believed that the presence of a charged contaminant will interact with the surface of the lipid particles and change the zeta potential of the mixture, and/or the average aggregate diameter of liposome aggregates in the mixture, compared to a control mixture.

As used herein a “charged contaminant” refers to a molecule that may be in the test composition and whose presence is being determined. A charged contaminant has a net positive or negative charge of +3 or greater (e.g., 3, 4, 5, 6, etc.), or −3 or less (e.g., −3, −4, −5, −6, etc.). The net positive or negative charge per molecule or per monomeric unit of a molecule (if such molecule is polymeric, e.g., includes repeating monomeric units), is referred to as charge density, and methods for determining the charge density of a molecule are known to the person skilled in the art.

In one embodiment, a charged contaminant is a polymer or includes a polymer. As used herein, a “polymer” refers to a molecule that includes at least two repeating units. There is no upper limit on the number of repeating units present in a charged contaminant detected using a method described herein.

The charge density of a polymer refers to the average net charge per repeating unit. Thus, a polysaccharide such as chondroitin sulfate is a chain of alternating sugars (N-acetylgalactosamine and glucuronic acid), and the charge density of chondroitin sulfate is the average net charge present on each repeating N-acetylgalactosamine and glucuronic acid disaccharide unit. The skilled person will recognize that a polymer may include additional charged groups attached to one or more repeating units. For instance, chondroitin sulfate will include sulfate groups. These additional charged groups are included when determining the average net charge per repeating unit. Since a charged contaminant has a charge density of +3 or greater or −3 or less, a polymer with a repeating unit having a charge density of +1 or −1 will have at least three repeating units.

An example of a polymer includes a polynucleotide, which is made up of repeated nucleotide monomers. A polynucleotide may be double stranded or single stranded, and may be DNA, RNA, or a combination thereof. Examples of charged contaminants that are polynucleotides include, but are not limited to, linear polynucleotides and circular polynucleotides (e.g., plasmids) that are in a relaxed state. A circular polynucleotide in a relaxed state is not over-wound or under-wound. An example of a circular polynucleotide that is not over-wound or under-wound is a plasmid that includes a nick in one strand. A circular polynucleotide that is not in a relaxed state is supercoiled. Whether a circular polynucleotide is in a relaxed state or supercoiled can be determined using methods known to the person skilled in the art and are routine.

Another example of a polymer is a polypeptide. As used herein, the term “polypeptide” refers broadly to two or more amino acids joined together by peptide bonds. The term “polypeptide” also includes molecules which contain more than one polypeptide joined by disulfide bonds, ionic bonds, or hydrophobic interactions, or complexes of polypeptides that are joined together, covalently or noncovalently, as multimers (e g, dimers, tetramers). Thus, the terms peptide, oligopeptide, and protein are all included within the definition of polypeptide and these terms are used interchangeably. In one embodiment, a polypeptide is linear or fibrous. A “linear” or “fibrous” polypeptide refers to a polypeptide that is not substantially globular. A “linear” or “fibrous” polypeptide may be a polypeptide that normally takes on a globular structure, but has been exposed to denaturing conditions that cause the globular structure to unwind and take on a more linear structure.

Another example of a polymer is a polysaccharide, which is made up of repeated saccharide units, e.g., repeated monosaccharide units, repeated disaccharide units, repeated trisaccharide units, etc. Examples of charged contaminants that are polysaccharides include, but are not limited to, glycosamainoglycans (such as dermatan sulfate, chondroitin sulfate, heparin, hyaluronic acid) and colanic acid (Grant et al., 1969, J. Bacteriol., 100(3):1187-1193). In one embodiment, a charged contaminant is a glycosaminoglycan that is over-sulfated or under-sulfated when compared to the analyte present in the test composition. In such an embodiment, examples of over-sulfated glycosaminoglycans include those which have a greater number of sulfate groups per disaccharide unit when compared with pharmaceutical-grade heparin. As used herein, “pharmaceutical-grade heparin” refers to heparin that is for clinical use in humans. Typically, pharmaceutical-grade heparin has a charge density of −3 per repeating disaccharide unit. Under-sulfated contaminants examples include contaminants which have fewer sulfate groups per disaccharide unit as compared with heparin, and thus have a lower charge density, such as heparan sulfate, dermatan sulfate, and hyaluronic acid.

In one embodiment, a charged contaminant includes a polymer. An example of a charged contaminant that includes a polymer includes, but is not limited to, lipopolysaccharide (LPS), a major constituent of the outer cell membrane of Gram-negative bacteria.

In one embodiment, a charged contaminant is an organic molecule. The charge density of an organic molecule refers to the overall charge of one organic molecule. An organic molecule may be a natural compound, i.e., a molecule produced by plants or animals, or a synthesized compound. Non-limiting examples of compounds include alkaloids, glycosides, nonribosomal peptides (such as actinomycin-D), phenazines, natural phenols (such as flavonoids), polyketides, terpenes (such as steroids), lipids (including lipid containing compounds), macrocycles, and tetrapyrroles.

A charged contaminant is soluble in an aqueous solution (a solution in which water is the solvent) or a semi-aqueous solution (a solution in which water is the primary solvent but one or more other solvents, such as an alcohol, is also present). In one embodiment, a charged contaminant is not a surfactant. As used herein, a “surfactant” is a compound that lowers the surface tension between two lipids. In one embodiment, a surfactant is a compound that disrupts the structure of lipid particle. In one embodiment a charged contaminant is a surfactant, but in such embodiments the concentration of the charged contaminant does not destabilize the lipid particles that are also used in the method.

A test composition may include more than one type of charged contaminant. For instance, a test composition may include one or more different organic molecules that are charged contaminants, one or more different polymers that are charged contaminants, or a combination of one or more different organic molecules and one or more polymers. When a test composition includes more than one type of charged contaminant, the charged contaminants may have difference charge densities. For instance, when a test composition includes two or more charged contaminants that are polymers and the analyte is heparin, one charged contaminant may be over-sulfated and another charged contaminant may be under-sulfated.

The test composition is an aqueous or semi-aqueous solution. The test composition can include any combination of compounds provided the compounds do not interfere with the ability to determine whether a charged contaminant is present. Accordingly, the concentration of ions cannot compete with or inhibit the interaction of a charged contaminant with lipid particles present in the test composition. Generally, the concentration of monovalent ions (ions having only a +1 or −1 charge) in solution is low, such as no greater than 10 mM, no greater than 5 mM, or no greater than 1 mM. In one embodiment, any monovalent ions in the test composition are undetectable using currently available detection methods. In those embodiments where a divalent and/or trivalent ion is present, the concentration of monovalent ions does not exceed 50%, does not exceed 40%, or does not exceed 30% of the concentration of divalent/trivalent ions in solution.

As used herein an “analyte” refers to the molecule that is present in the test composition and whose level of purity with respect to charged contaminants is being determined using the methods described herein. An analyte is miscible in the aqueous or semi-aqueous solution. In one embodiment, an analyte is not a surfactant, and in one embodiment an analyte is a surfactant, but is present in a concentration that does not destabilize the lipid particles that are also used in the method. In one embodiment, an analyte does not have a viscosity that inhibits the ability to detect changes in zeta potential and/or average aggregate diameter in a mixture. A test composition may include more than one analyte.

The methods described herein are not intended to be limited by the analyte present in a test composition. Thus, an analyte is any molecule provided it has the characteristics discussed herein (e.g., it is miscible in the aqueous or semi-aqueous solution). Analytes include, but are not limited to, polymers and organic molecules, such as the polymers and organic molecules described above as examples of charged contaminants. In other words, a compound can be a charged contaminant in one situation, and an analyte in another. In one embodiment, the difference in charge density between the charged contaminant and the analyte is at least +/−2 for an organic molecule, and +/−3 for a polymer. For instance, if the repeating unit of a polymer has a charge density of +1 or −1, then the polymer will have at least three repeating units. In one embodiment there is no difference in charge density between the charged contaminant and the analyte.

In one embodiment, an analyte is a glycosaminoglycan product. Examples of glycosaminoglycan products include, but are not limited to, heparin preparations, supplement-grade chondroitin, and various glycosaminoglycans such as those used for research purposes. In one embodiment, when an analyte is a glycosaminoglycan product, a charged contaminant being tested includes over-sulfated glycosaminoglycans, under-sulfated glycosaminoglycans, or both. For instance, in one embodiment where the analyte is heparin, a method described herein can be used to detect the presence of over-sulfated glycosaminoglycans, under-sulfated glycosaminoglycans, such as dermatan sulfate (also known as chondroitin sulfate B) and chondroitin sulfate, or both over-sulfated and under-sulfated glycosaminoglycans.

In one embodiment, an analyte is a double stranded circular polynucleotide, such as a plasmid, and the charged contaminant is a polynucleotide, either linear or circular, that is in a relaxed state. When DNA is supercoiled, it becomes denser, and takes on a more compact form. Without intending to be limited by theory, it is expected that the interaction of supercoiled DNA with the surface of the lipid particles will be considerably weaker than in DNA in a relaxed state.

In one embodiment, the analyte is present in a test composition such that the final concentration of the analyte, or combination of analytes, in the mixture is at least 0.1 milliMolar (mM), at least 1 mM, at least 10 mM, at least 100 mM, at least 200 mM, at least 300 mM, at least 400 mM, at least 500 mM, at least 600 mM, at least 700 mM, at least 800 mM, at least 900 mM, or at least 1 M. In one embodiment, the analyte is present in a test composition such that the final concentration of analyte, or combination of analytes, in the mixture is no greater than 800 milliMolar (mM), no greater than 700 mM, no greater than 600 mM, no greater than 500 mM, no greater than 400 mM, no greater than 300 mM, no greater than 200 mM, no greater than 100 mM, no greater than 10 mM, no greater than 1 mM, or no greater than 0.1 mM. In one embodiment, the analyte, or combination of analytes, is present in a test composition such that the final concentration of analyte, or combination of analytes, in the mixture is a range between at least 0.1 mM and no greater than 800 mM, or any combination of concentrations selected from the numbers listed above.

In one embodiment, the analyte is present in a test composition such that the final concentration of the analyte, or combination of analytes, in the mixture is at least 1 micrograms per mL (μg/mL), at least 10 μg/mL, at least 100 μg/mL, at least 200 μg/mL, at least 300 μg/mL, at least 400 μg/mL, at least 500 μg/mL, at least 600 μg/mL, at least 700 μg/mL, at least 800 μg/mL, at least 900 μg/mL, or at least 1000 μg/mL In one embodiment, the analyte is present in a test composition such that the final concentration of analyte, or combination of analytes, in the mixture is no greater than 2000 μg/mL, no greater than 1000 μg/mL, no greater than 900 μg/mL, wno greater than 800 μg/mL, no greater than 700 μg/mL, no greater than 600 μg/mL, no greater than 500 μg/mL, no greater than 400 μg/mL, no greater than 300 μg/mL, no greater than 200 μg/mL, no greater than 100 μg/mL, no greater than 10 μg/mL, or no greater than 1 μg/mL. In one embodiment, the analyte, or combination of analytes, is present in a test composition such that the final concentration of analyte, or combination of analytes, in the mixture is a range between at least 500 μg/mL and no greater than 2000 μg/mL, or any combination of concentrations selected from the numbers listed above.

As used herein, a “lipid particle” is a structure that self-assembles in aqueous solutions and includes amphipathic molecules. In one embodiment, a lipid particle is approximately spherical in shape.

As used herein, an “amphipathic” molecule is one that has both hydrophilic and hydrophobic properties. An amphipathic molecule has hydrophilic properties and hydrophobic properties, and in one embodiment an amphipathic molecule has the hydrophilic properties and hydrophobic properties at separate ends of the molecules. The hydrophilic properties may be due to functional groups, either ionic or uncharged. Examples of ionic groups include, but are not limited to, anionic groups such as carboxylates, sulfates, sulfonates, and phosphates, and cationic groups such as amines Examples of uncharged groups include, but are not limited to, alcohols. The hydrophilic end of an amphipathic molecule may be a zwitterion, positively charged, or negatively charged.

The hydrophobic properties of an amphipathic molecule may be due to a hydrocarbon chain, such as one in the form of CH₃(CH₂)ₙ, with n greater than 2 In one embodiment, n is no greater than 25. An amphipathic molecule may include 1, 2, or 3 hydrocarbon chains, and each chain may be independently saturated or include unsaturated carbon-carbon bonds. In one embodiment, the number of unsaturated bonds may be 1, 2, 3, 4, 5, or 6. In one embodiment, the number of unsaturated bonds may be between 25% and 75% of the hydrocarbon chain, or between 40% and 60% of the hydrocarbon chain. Examples of amphipathic molecules include, but are not limited to, phospholipids; sphingolipids, such as sphingosines, phosphosphingolipids, and ceramides; and block amphipathic copolymers.

Examples of lipid particles include, but are not limited to, micelles, liposomes, and polymersomes. A micelle is a structure that has hydrophilic head regions of the amphipathic molecules on the exterior and interacting with a surrounding aqueous solvent and has the hydrophobic regions of the amphipathic molecules present in the center of the structure. In one embodiment, an amphipathic molecule present in a micelle may have one hydrocarbon chain A liposome is a structure that includes a lipid bilayer that encloses an aqueous interior compartment. The lipid bilayer of a liposome typically includes at least one type of phospholipid. A polymersome is a structure that encloses an interior compartment and may have the bilayer morphology of a liposome or of a micelle, but is made up of block copolymer amphiphiles.

A population of lipid particles used in a method described herein may have a diameter of between 20 nanometers (nm) and 1 micron, and all numbers subsumed within that range. In one embodiment, the lipid particles have an average diameter that is at least 20 nm, at least 40 nm, at least 50 nm, at least 100 nm, at least 200 nm, at least 300 nm, at least 400, at least 500 nm, or at least 600 nm. In one embodiment, the liposomes have a diameter of no greater than 1 micron, no greater than 900 nm, no greater than 800 nm, no greater than 700 nm, no greater than 600 nm, no greater than 500 nm, no greater than 400 nm, no greater than 300 nm, no greater than 200 nm, no greater than 100 nm, or no greater than 50 nm. In one embodiment, the lipid particle, such as a liposome, has a diameter of between 150 nm and 250 nm. In one embodiment, the lipid particle, such as a micelle, has a diameter of between 20 nm and 1000 nm. In one embodiment, such as where a small organic molecule is a charged contaminant, the lipid particle, such as a micelle, has a diameter of between 20 nm and 100 nm.

In one embodiment, the lipid particles, such as micelles, are made up of lipids having a single tail. Examples of such lipids include, but are not limited to, phosphorylated sphingosines, such as D-erythro-sphingosine-1-phosphate.

In one embodiment, the lipid particles, such as liposomes, are made up of phospholipids which include two hydrocarbon chains. A phospholipid present in a lipid particle may have both hydrocarbon chains saturated, both hydrocarbon chains unsaturated, or one chain saturated and one chain unsaturated. In one embodiment, any combination of two more such phospholipids may be present in a liposome. In one embodiment, a lipid particle, such as a liposome, includes phospholipids having one saturated hydrocarbon chain and one unsaturated hydrocarbon chain having one double bond. In one embodiment, the concentration in the liposome of phospholipids having one unsaturated hydrocarbon chain and one saturated hydrocarbon chain, two unsaturated hydrocarbon chains, two saturated hydrocarbon chains, or a combination thereof, may be between 95 mol % and 100 mol %, and all numbers subsumed within that range, for instance, 96 mol %, 97 mol %, 98 mol %, 99 mol %, and 99.5 mol %. In one embodiment, lipid particles, such as liposomes, may include other lipids that are not phospholipids, such as, but not limited to, cholesterol.

Examples of phospholipids having one or two unsaturated hydrocarbon chains include, but are not limited to, POPC (1-palmitoyl-2-oleoyl-sn-glycero-3-phosphocholine), SOPC (1-stearoyl-2-oleoyl-sn-glycero-3-phosphocholine), OSPC (1-oleoyl-2-stearoyl-sn-glycero-3-phosphocholine), OPPC (1-oleoyl-2-palmitoyl-sn-glycero-3-phosphocholine), DOPC (1,2-dioleoyl-sn-glycero-3-phosphocholine), OMPC (1-oleoyl-2-myristoyl-sn-glycero-3-phosphocholine), 1,2-dipalmitoleoyl-sn-glycero-3-phosphocholine, 1,2-dieicosenoyl-sn-glycero-3-phosphocholine, 1,2-dierucoyl-sn-glycero-3-phosphocholine, 1,2-dinervonoyl-sn-glycero-3-phosphocholine, Egg PC (L-α-phosphatidylcholine (Egg, Chicken)), DOPE (1,2-dioleoyl-sn-glycero-3-phosphoethanolamine), and 1,2-dipalmitoleoyl-sn-glycero-3-phosphoethanolamine. In one embodiment, a combination of two or more such phospholipids may be present in a lipid particle.

Examples of phospholipids having two saturated hydrocarbon chains include, but are not limited to, DMPC (1,2-dimyristoyl-sn-glycero-3-phosphocholine), MPPC (1-myristoyl-2-palmitoyl-sn-glycero-3-phosphocholine), MSPC (1-myristoyl-2-stearoyl-sn-glycero-3-phosphocholine), DPPC (1,2-dipalmitoyl-sn-glycero-3-phosphocholine), PSPC (1-palmitoyl-2-stearoyl-sn-glycero-3-phosphocholine), DSPC (1,2-distearoyl-sn-glycero-3-phosphocholine), 1,2-diheptadecanoyl-sn-glycero-3-phosphocholine, 1,2-dipentadecanoyl-sn-glycero-3-phosphocholine, 1,2-dinonadecanoyl-sn-glycero-3-phosphocholine, 1,2-diarachidoyl-sn-glycero-1,2-dihenarachidoyl-sn-glycero-3-phosphocholine, 1,2-dibehenoyl-sn-glycero-3-phosphocholine, 1,2-ditricosanoyl-sn-glycero-3-phosphocholine, 1,2-dilignoceroyl-sn-glycero-3-phosphocholine, DMPE (1,2-dimyristoyl-sn-glycero-3-phosphoethanolamine), DPPE (1,2-dipalmitoyl-sn-glycero-3-phosphoethanolamine), DSPE (1,2-distearoyl-sn-glycero-3-phosphoethanolamine), 1,2-dipentadecanoyl-sn-glycero-3-phosphoethanolamine, 1,2-diheptadecanoyl-sn-glycero-3-phosphoethanolamine, and 1,2-dipalmitoyl-sn-glycero-3-phosphoethanolamine-N-(lissamine rhodamine B sulfonyl). In one embodiment, a combination of two or more such phospholipids may be present in a lipid particle.

In one embodiment, the lipid particles include, or are made up of, amphipathic molecules having a positively charged hydrophilic region. Examples of such amphipathic molecules include, but are not limited to, 1,2-di-O-octadecenyl-3-trimethylammonium propane, 1,2-dilauroyl-sn-glycero-3-ethylphosphocholine, 1,2-dimyristoyl-sn-glycero-3-ethylphosphocholine, 1,2-dimyristoleoyl-sn-glycero-3-ethylphosphocholine, 1,2-dipalmitoyl-sn-glycero-3-ethylphosphocholine, 1,2-distearoyl-sn-glycero-3-ethylphosphocholine, 1,2-dioleoyl-sn-glycero-3-ethylphosphocholine, 1-palmitoyl-2-oleoyl-sn-glycero-3-ethylphosphocholine, N1-[2-((1S)-1-[(3-aminopropyl)amino]-4-[di(3-amino-propyl)amino]butylcarboxamido)ethyl]-3,4-di[oleyloxy]-benzamide (also referred to as MVL5), Dimethyldioctadecylammonium, 1,2-dioleoyl-3-trimethylammonium-propane, 1,2-dimyristoyl-3-trimethylammonium-propane, 1,2-dipalmitoyl-3-trimethylammonium-propane, and 1,2-stearoyl-3-trimethylammonium-propane. In one embodiment, a lipid particle including one or more amphipathic molecules having a positively charged hydrophilic region is a liposome. In one embodiment, a combination of two more such amphipathic molecules may be present in a lipid particle.

The skilled person will appreciate that the lipids that make up a lipid particle may influence the conditions used to determine whether a test composition includes a charged contaminant. For instance, in some embodiments, when positively charged lipids are used the inclusion of divalent or trivalent cations in the test composition is less desirable. Likewise, in some embodiments, when zwitterionic lipids are used the inclusion of divalent or trivalent cations in the test composition is more desirable. The skilled person will also appreciate that the use of certain the lipids in a lipid particle may be more desirable depending upon the analyte present in the test composition and/or the charged contaminant that may be present in the test composition. For instance, in an embodiment where the analyte includes heparin and the charged contaminant is an over- or under-sulfated glycosaminoglycan, the phospholipids of a lipid particle include one having at least one chain that is unsaturated and present at a concentration of at least 99 mol %; however, other lipids and other concentrations are also useful for determining the presence of over- or under-sulfated glycosaminoglycans in a composition that includes a heparin analyte. In one embodiment, lipid particles having positively charged amphipathic molecules are useful when the analyte is supercoiled DNA or RNA and the charged contaminant is relaxed DNA or RNA. In one embodiment, lipid particles that include POPC, DSPC, or the combination thereof, may be used when the charged contaminant includes LPS.

In one embodiment, the lipid particles include, or are made up of, block amphipathic copolymers. Examples of block amphipathic copolymers are known and readily produced by the skilled person (see Brinkhuis et al., 2011, Polym. Chem., 2:1449-1462).

In one embodiment, a method described herein further includes supplementing the mixture with an ion. The ion may be monovalent or multivalent (e.g., divalent or a trivalent), and may be a cation or anion. Examples of divalent cations include, but are not limited to, Magnesium (Mg++), Zinc (Zn++), and Calcium (Ca++). Examples of trivalent cations include, but are not limited to, Lanthanum (La+++) and Cerium (Ce+++). In one embodiment, any combination of two more cations or two or more anions may be present in a mixture. In one embodiment, the final concentration of cations or anions in a mixture may be at least 100 micromolar (uM), at least 300 uM, at least 500 uM, at least 700 uM, at least 900 uM, at least 1 mM, at least 5 mM, at least 10 mM, at least 20 mM, at least 30 mM, at least 40 mM, at least 50 mM, at least 60 mM, at least 70 mM, at least 80 mM, at least 90 mM, or at least 100 mM. In one embodiment, the mixture may be supplemented when the lipids used to form the nanoparticles are zwitterionic.

In one embodiment, a method described herein further includes adding to the test composition an enzyme to alter the characteristics of the test composition and ease the identification of a charge contaminant. For instance, when the test composition includes polynucleotides, such as genomic DNA, and the method is for determining the presence of a non-polynucleotide contaminant, an exonuclease and/or endonuclease may be added to the test composition to decrease the degree of polymerization of the polynucleotides. Removal of polynucleotides such a genomic DNA may be useful when the viscosity of the solution is high and the result of polynucleotides. In one embodiment, a nuclease may be used when the analyte has been produced by a cell, such as a eukaryotic or prokaryotic cell. In one embodiment, a nuclease may be used when the analyte has been produced by a gram negative microbe and the charged contaminant is LPS.

In one embodiment, a method described herein further includes processing a test composition to increase the sensitivity of the method. In one embodiment, such as those embodiments where the analyte is a polymer, including a charged polymer, the processing results in depolymerizing the analyte and not altering the characteristics of the charged contaminant. For instance, where the analyte includes heparin, the method may further include exposing the heparin to conditions that reduce the size of the heparin. In one embodiment, the size of the heparin molecules is reduced by digestion with a heparinase, such as heparinase I, heparinase II, and/or heparinase III. Methods for using a heparinase to digest heparin are known and routine. In one embodiment, the size of the heparin molecules is reduced by exposure to nitrous acid.

The methods described herein include comparing characteristics of the mixture to a control mixture. A control mixture is a mixture that is identical to the mixture except for the charged contaminant Thus, a control mixture includes the lipid particles at the same concentration as the mixture with the charged contaminant, and the analyte at the same concentration as the mixture with the charged contaminant. The analyte in the control mixture is at a known level of purity with respect to charged contaminants. In general, having less charged contaminants present in the control mixture will increase the sensitivity of the assay for charged contaminants in the mixture. If the mixture being assayed includes, for instance, added cations, nucleases, heparinases, nitrous acid, or any other component, the control mixture may also, and in some embodiments does, include the added components. The level of purity of an analyte in a control mixture may be determined using routine and known, but generally time consuming, methods. For instance, heparin standard of known purity may be obtained by testing a commercial heparin preparation using known techniques for measuring contaminants, including, for instance, ¹H NMR spectroscopy and/or string anion exchange HPLC.

In one embodiment, the method includes determining the zeta potential of the mixture and comparing it to the zeta potential of a control mixture. Methods for determining zeta potential of a mixture are known in the art and are routine. Typically, methods for determining zeta potential include, but are not limited to, mobilitylaser Doppler velocimetry and phase analysis light scattering.

In one embodiment, the method includes determining the average aggregate diameter of liposome aggregates in the mixture and comparing it to the average aggregate diameter of liposome aggregates in a control mixture. Methods for determining average aggregate diameter of liposome aggregates in a mixture are known in the art and are routine. A preferred example of a method is dynamic light scattering, as disclosed herein in Example 1.

The detection of a difference in zeta potential and/or average aggregate diameter of liposome aggregates between the mixture and the control mixture indicates the presence of a charged contaminant.

In one embodiment, the difference between the zeta potential of the mixture and the zeta potential of the control mixture, and/or the difference between the average aggregate diameter of liposome aggregates in the mixture and the average aggregate diameter of liposome aggregates in the control mixture, is statistically significant. The difference may be evaluated using known methods of statistical analysis. In one embodiment, a statistically significant change is a change at α=0.05 using standard Student's T-test. In one embodiment, the presence of one or more charged contaminants results in a drop in zeta potential and/or an increase of average aggregate diameter of at least 5%, at least 10%, at least 20%, at least 30%, at least 40%, or at least 50% compared to the control mixture.

In one embodiment, a method described herein has the ability to detect charged contaminants that are present in the test composition at a level of at least 0.3% weight of charged contaminant(s)/weight of analyte(s) (w/w), at least 0.5% w/w, at least 1% w/w, at least 3% w/w, or at least 5% w/w.

Also provided herein are methods for enriching analytes from contaminants present in solution. Without intending to be limited in any way by theory, it is believed that under certain conditions an analyte can interact with lipid particles to a greater degree than contaminants present in the mixture, and then steps can be taken to separate the analyte/lipid particle complex from contaminants. As used herein, the term “enriched” means that the amount of an analyte relative to the amount of one or more contaminants has been increased at least 2 fold, at least 5 fold, at least 10 fold, or at least 15 fold. Enrichment does not imply that all contaminants have been removed.

The method includes combining a test composition with lipid particles and cations to form a mixture, and incubating the mixture under conditions suitable for forming a complex that includes the analyte bound to the lipid particle. The test composition includes at least one analyte and at least one contaminant. In one embodiment, the difference in charge density between the charged contaminant and the analyte is at least +/−2 for an organic molecule, and +/−3 for a polymer.

The test composition is an aqueous or semi-aqueous solution. The test composition can include any combination of compounds provided the compounds do not interfere with the ability of an analyte to interact with a lipid particle and form a complex.

In this method an “analyte” refers to the molecule that is present in the test composition and is being removed from contaminants also present in the test composition. An analyte is miscible in the aqueous or semi-aqueous solution. In one embodiment, an analyte is not a surfactant, and in one embodiment an analyte is a surfactant, but is present in a concentration that does not destabilize the lipid particles that are also used in the method. In one embodiment, an analyte does not have a viscosity that inhibits the ability of the analyte and lipid particles to interact. A test composition may include more than one analyte. Examples of such analytes include, but are not limited to, polynucleotides, including DNA and RNA molecules, and glycosaminoglycans. In one embodiment, such as when the analyte is a polynucleotide, the concentration of analyte is, is at least, or is no greater than, 0.5 mg/ml, 1 mg/mL, 4 mg/mL, or 8 mg/mL. In one embodiment, such as when the analyte is a glycosaminoglycan, the concentration of analyte is, is at least, or is no greater than, 5 mg/ml, 10 mg/mL, or 15 mg/mL.

In this method a contaminant is a molecule present in the test composition that is to be separated from the analyte. A contaminant has a net positive or negative charge density that is less than the charge density of the analyte. In one embodiment, the difference in charge density between the contaminant and the analyte is at least +/−1 to +/−2 for an organic molecule, and +/−2 for a polymer. In one embodiment, the analyte is charged over at least 75%, at least 85%, at least 95%, or 100% of the molecule, while the contaminant would is charged over no greater than 25%, no greater than 15%, no greater than 5% of the molecule, or has no charge (e.g., when the analyte is DNA and the contaminant includes colanic acid. Examples of such contaminants include, but are not limited to, organic molecules and polymers. Examples of polymers include, but are not limited to, LPS and colanic acid.

The cations present in the mixture are multivalent, e.g., divalent or a trivalent. Examples of divalent cations include, but are not limited to, Magnesium (Mg++), Zinc (Zn++), and Calcium (Ca++). Examples of trivalent cations include, but are not limited to, Lanthanum (La+++) and Cerium (Ce+++). In one embodiment, any combination of two more cations may be present in a mixture. In one embodiment, the final concentration of cations in a mixture may be at least 80 mM, at least 90 mM, at least 100 mM, at least 110 mM, at least 120 mM, at least 130 mM, at least 140 mM, or at least 150 mM.

In one embodiment, the lipid particles are liposomes. In one embodiment, the lipid particles present in the mixture include phospholipids having two saturated hydrocarbon chains. Examples of phospholipids having two saturated hydrocarbon chains include, but are not limited to, DMPC (1,2-dimyristoyl-sn-glycero-3-phosphocholine), MPPC (1-myristoyl-2-palmitoyl-sn-MSPC (1-myristoyl-2-stearoyl-sn-glycero-3-phosphocholine), DPPC (1,2-dipalmitoyl-sn-glycero-3-phosphocholine), PSPC (1-palmitoyl-2-stearoyl-sn-glycero-3-phosphocholine), DSPC (1,2-distearoyl-sn-glycero-3-phosphocholine), 1,2-diheptadecanoyl-sn-glycero-3-phosphocholine, 1,2-dipentadecanoyl-sn-glycero-3-phosphocholine, 1,2-dinonadecanoyl-sn-glycero-3-phosphocholine, 1,2-diarachidoyl-sn-glycero-3-phosphocholine, 1,2-dihenarachidoyl-sn-glycero-3-phosphocholine, 1,2-dibehenoyl-sn-glycero-3-phosphocholine, 1,2-ditricosanoyl-sn-glycero-3-phosphocholine, 1,2-dilignoceroyl-sn-glycero-3-phosphocholine, DMPE (1,2-dimyristoyl-sn-glycero-3-phosphoethanolamine), DPPE (1,2-dipalmitoyl-sn-glycero-3-phosphoethanolamine), DSPE (1,2-distearoyl-sn-glycero-3-phosphoethanolamine), 1,2-dipentadecanoyl-sn-glycero-3-phosphoethanolamine, 1,2-diheptadecanoyl-sn-glycero-3-phosphoethanolamine, and 1,2-dipalmitoyl-sn-glycero-3-phosphoethanolamine-N-(lissamine rhodamine B sulfonyl). In one embodiment, a combination of two or more such phospholipids may be present in a lipid particle.

The method optionally includes separating the complex from the contaminant. As the complex and contaminant are present in solution, known methods for separating the heavier complex may be used. Examples of methods include, but are not limited to, centrifugation.

The method optionally includes separating the complex into analyte and lipid particle. This separation may be accomplished by exposing the complex to a solution of low ionic strength, such as deionized water. The heavier lipid particles can then be removed using know methods, such as centrifugation.

The present invention is illustrated by the following examples. It is to be understood that the particular examples, materials, amounts, and procedures are to be interpreted broadly in accordance with the scope and spirit of the invention as set forth herein.

### Example 1

### Glycosaminoglycan-Mediated Selective Changes in the Aggregation States, Zeta Potentials, and Intrinsic Stability of Liposomes

Though the aggregation of glycosaminoglycans (GAGs) in the presence of liposomes and divalent cations has been previously reported, the effect of different GAG species, as well as minor changes in GAG composition on the aggregates formed is yet unknown. If minor changes in GAG composition produce observable changes in liposome aggregate diameter or zeta potential, such a phenomenon may be used to detect potentially dangerous over-sulfated contaminants in heparin. We studied the mechanism of the interactions between heparin and its over-sulfated glycosaminoglycan contaminants with liposomes. Herein, we demonstrate that Mg²⁺ acts to shield the incoming glycosaminoglycans from the negatively-charged phosphate groups of the phospholipids, and that changes in the aggregate diameter and zeta potential are a function of glycosaminoglycan species and concentration, as well as liposome bilayer composition. These observations are supported by TEM studies. We have shown that organizational states of the liposome bilayers are influenced by the presence of GAG and excess Mg²⁺, resulting in a stabilizing effect which increases the Tₘ value of DSPC liposomes; the magnitude of this effect is also dependent on GAG species and concentration present. There is an inverse relationship between the percent change of aggregate diameter and percent change of aggregate zeta potential, as a function of GAG concentration in solution. Finally, we demonstrate that the diameter and zeta potential changes of POPC liposome aggregates in the presence of different over-sulfated heparin contaminants at low concentrations allow accurate detection of over-sulfated chondroitin sulfate at concentrations as low as 1 mol %.

### Materials and Methods

Materials and synthesis of over-sulfated GAGs: Chondroitin-6-sulfate, dermatan sulfate, and heparin were sourced from Spectrum Chemical Corp., CalBiochem, and Alfa Aesar, respectively. Each was over-sulfated using the procedures published by Maruyama, et al³ and Nagasawa, et al²⁰.

Preparation of liposomes for aggregation: Stock solutions of 1-palmitoyl-2-oleoyl-sn-glycero-3-phosphocholine (POPC, commercially available from Avanti Polar Lipids, Alabaster, Ala.) and 1,2-distearoyl-sn-glycero-3-phosphocholine (DSPC, commercially available from Avanti Polar Lipids, Alabaster, Ala.) were prepared in chloroform at a concentration of 2 mg/mL Stock solution of 1,2-dipalmitoyl-sn-glycero-3-phosphoethanolamine-N-(lissamine rhodamine B sulfonyl) (ammonium salt) (rhodamine lipid, commercially available from Avanti Polar Lipids, Alabaster, Ala.) was prepared in chloroform at a concentration of 0.01 mg/mL Stock solution of pyranine lipid was prepared in chloroform at a concentration of 0.01 mg/mL Lipid mixtures containing POPC were obtained by combining 2.4 mL POPC stock solution, and either 8.0 mL rhodamine lipid stock solution or 8.4 mL pyranine lipid stock solution. Mixtures containing DSPC were prepared by combining 2 mL DSPC stock solution and 6.5 mL rhodamine lipid stock solution.

The resulting mixtures had molar ratios of 99:1 POPC (or DSPC):rhodamine lipid/pyranine lipid, respectively. The mixture was subjected to rotary evaporation at 50° C. for 15 minutes, forming a thin film adhering to the sides of the flask. This thin film was then dried overnight under high vacuum to ensure complete removal of solvent. Lipid films containing POPC as the main lipid were then hydrated with 4.0 mL of 25 mM HEPES buffer at pH 8 by rapid rotation in a 50° C. water bath for 1 hr. Lipid films containing DSPC as the main lipid were hydrated with 4.0 mL of 25 mM HEPES buffer at pH 8 by rapid rotation in a 70° C. water bath for 1 hr. The procedure now varies for production of 50 nm, 200 nm, and 500 nm liposomes:
For 50 nm diameter liposomes (POPC liposomes only): the hydrated solution was probe sonicated at 70° C. for 45 minutes, followed by extrusion at 70° C. (15 times) through polycarbonate membrane filters (100 nm pore size). The average diameter of the prepared liposomes (by DLS) was approximately 55 nm+25 nm.
For 200 nm diameter liposomes (POPC and DSPC liposomes): the hydrated solution was immediately extruded at 70° C. (15 times) through polycarbonate membrane filters (100 nm pore size). Average measured diameters (by DLS) were approximately 185±8 nm and 250±90 nm for POPC and DSPC liposomes, respectively.
For 550 nm diameter liposomes (POPC liposomes only): Following hydration, the resulting large vesicles were found to have an average diameter of 550±70 nm (by DLS). These liposomes were used as such.
   The final volume of each respective liposome solution was then measured using the extrusion syringes, and the total lipid per unit volume calculated from this volume. All liposome solutions were diluted to 1.4 mM total lipid before use.

Mechanistic studies—influence of GAG species and Mg²⁺ on diameter and zeta potential of aggregates: DSPC or POPC liposomes (200 nm diameter only) were incubated for 15 minutes at room temperature in the presence and absence of Mg²⁺ (33.4 mM final concentration), as well as the presence and absence of heparin. Mixtures were produced according to Table 1 below.

TABLE 1
Preparation of liposomes for diameter and zeta
potential mechanism tests (volume in μL)

| | HEPES | Liposomes | MgSO₄ | GAG |
|---|---|---|---|---|
| | buffer (25 | (1.4 mM | (2M in | (1 μM in |
| | mM, pH 8) | total lipid) | HEPES) | HEPES) |
| Liposomes only | 306 | 50 | — | — |
| Liposomes + GAG | 246 | 50 | — | 60 |
| Liposomes + Mg²⁺ | 300 | 50 | 6 | — |
| Liposomes + | 240 | 50 | 6 | 60 |
| Mg²⁺ + GAG | | | | |

Each mixture was allowed to incubate at room temperature for 15 minutes before reading. One hundred μL of this aggregated solution was mixed with 900 μL HEPES buffer at pH 8 in a disposable polystyrene cuvette, and read on a Malvern Zetasizer Nano ZS90 with the following settings: 5 measurements, each an average of 10 reads, each read 10 sec; 90° read angle; 60 second pre-equilibration; Auto Attenuation off, manual attenuation set to 7. For the corresponding zeta potential measurements, liposomes were aggregated in the same way as above. Zeta potential was read on a Malvern Zetasizer Nano ZS90 with the following settings: 5 measurements, each an average of 10 reads, each read 10 seconds; 60 second pre-equilibration; automatic attenuation on; automatic voltage selection on.

Mechanistic studies—influence of GAG species and concentration on the saturation of aggregate diameter and zeta potential: For tests with individual GAGs, POPC and DSPC liposomes were aggregated in the presence of eight different concentrations of each GAG (heparin, over-sulfated chondroitin sulfate, over-sulfated dermatan sulfate, or over-sulfated heparin) in preparation for DLS, according to Table 2 below.

TABLE 2
Preparation of liposome aggregates for
saturation tests (all volumes in μL)

| | HEPES | Liposomes | | GAG | |
|---|---|---|---|---|---|
| | buffer (25 | (1.4 mM | MgSO₄ | (concentration | |
| | mM, pH 8) | total lipid) | at 2M | in parentheses) | |
| Liposomes | 300 | 50 | 6 | — | |
| only + Mg²⁺ | | | | | |
| 100 nM GAG | 264 | 50 | 6 | 35.6 | (1 μM) |
| 500 nM GAG | 122 | 50 | 6 | 178 | (1 μM) |
| 1 μM GAG | 296 | 50 | 6 | 3.6 | (100 μM) |
| 10 μM GAG | 264 | 50 | 6 | 35.6 | (100 μM) |
| 50 μM GAG | 122 | 50 | 6 | 178 | (100 μM) |
| 100 μM GAG | 264 | 50 | 6 | 35.6 | (1 mM) |
| 250 μM GAG | 211 | 50 | 6 | 89 | (1 mM) |
| 500 μM GAG | 122 | 50 | 6 | 178 | (1 mM) |

Measurement of aggregate diameter and zeta potential proceeded in the same way as stated above. Three measurements were collected for each GAG concentration for both average diameter and zeta potential, each an average of 10 reads, each read 10 seconds. Equipment settings remained the same.

TEM imaging: To aggregate liposomes, 50 μL of liposomes (200 nm diameter) at 1.4 mM, were incubated with 60 μL of GAG at 1 μM (approximately 20% v/v, 170 nM final concentration) and 6 μL of MgSO₄ at 2 M in 240 μL HEPES buffer at pH 8 for 15 minutes at room temperature. For liposome only control, 60 μL GAG was substituted with 60 μL additional HEPES buffer. Copper TEM grids (300-mesh, formvar-carbon coated, Electron Microscopy Sciences, Hatfield, Pa., USA) were prepared by applying a drop of 0.01% poly-L-lysine, allowing it to stand for 30 seconds, wicking off the liquid with torn filter paper, and allowing the grids to air dry. A drop of the aggregated liposome suspension was placed on a prepared grid for 30 seconds and wicked off; grids were allowed to air dry again. Phosphotungstic acid 1%, pH adjusted to 7-8, was dropped onto the grid containing the liposome sample, allowed to stand for 1.5 min, and wicked off. After the grids were dry, images were obtained using a JEOL JEM-2100 LaB₆ transmission electron microscope (JEOL USA, Peabody, Mass.) running at 200 keV.

Differential scanning calorimetry: DSPC liposomes were incubated with 1 μM and 250 μM GAG for 15 minutes at room temperature, before being degassed for 15 minutes and loaded into a Nano DSC (TA instruments New Castle, Del.) without further dilution. A sample of DSPC liposomes incubated with only Mg²⁺ was used as the control. The DSC reference cell was filled with HEPES buffer at 25 mM, pH 8, containing 33.4 mM MgSO₄, the same as that of the samples. Machine was pressurized to three atmospheres, and scans were conducted from 25° C. to 75° C., and rate of temperature change was 2° C./minute. Heat required during transition was calculated using NanoAnalyze software provided by the instrument vendor, using the sigmoidal baseline function to produce the pre- and post-transition baseline.

Mechanistic studies—combined influence of liposome diameter and GAG concentration on diameter and zeta potential changes: POPC liposomes of diameters 50, 200, and 550 nm diameter liposomes were each incubated with heparin, OSH, OSCS, and OSD (individually) at concentrations of 50, 170, and 500 nM. Measurement of aggregate diameter and zeta potential were measured in the same way as stated above. Five measurements were collected for each GAG concentration for both diameter and zeta potential, each an average of 10 reads, each read 10 seconds. Equipment settings remained the same. Following collection of data, each over-sulfated contaminant was compared to the corresponding measurement of heparin by calculating the percent change from heparin, using the following formula:

$$( \frac{size of contaminant aggregate - size of heparin aggregate}{size of heparin aggregate} ) \times 100$$

The same formula was applied to calculate zeta potential percent change.

Heparin contamination studies: For contaminated heparin studies, final concentrations of 170 nM and 500 nM total GAG were used with 200 nm and 500 nm diameter liposomes, respectively. Solutions of heparin with an over-sulfated contaminant were prepared according to Tables 3 and 4 below.

TABLE 3
Preparation of liposome aggregates for 170
nM contamination study (all volumes in μL)

| | HEPES buffer | Liposomes (1.4 | MgSO₄ | Heparin (1 μM | Over-sulfated | |
|---|---|---|---|---|---|---|
| | (25 mM, pH 8) | mM total lipid) | at 2M | concentration) | contaminant | |
| Heparin only | 240 | 50 | 6 | 60 | — | |
| 0.5 mol % | 237.3 | 50 | 6 | 59.7 | 3 | (100 nM) |
| contamination | | | | | | |
| 1.0 mol % | 234.3 | 50 | 6 | 59.4 | 6 | (100 nM) |
| contamination | | | | | | |
| 2.5 mol % | 240 | 50 | 6 | 58.5 | 1.5 | (1 μM) |
| contamination | | | | | | |
| 5.0 mol % | 240 | 50 | 6 | 57 | 3 | (1 μM) |
| contamination | | | | | | |
| 10.0 mol % | 240 | 50 | 6 | 54 | 6 | (1 μM) |
| contamination | | | | | | |
| 15.0 mol % | 240 | 50 | 6 | 51 | 9 | (1 μM) |
| contamination | | | | | | |
| 20.0 mol % | 240 | 50 | 6 | 48 | 12 | (1 μM) |
| contamination | | | | | | |
| 30.0 mol % | 240 | 50 | 6 | 42 | 15 | (1 μM) |
| contamination | | | | | | |

Measurement of aggregate diameter and zeta potential proceeded in the same way as stated above. Five measurements were collected for each GAG concentration for both diameter and zeta potential, each an average of 10 reads, each read 10 seconds. Equipment settings remained the same.

Statistical analysis: Analysis of variance and Dunnett's post-tests were run using Minitab software, version 16.1.1.

TABLE 4
Preparation of liposome aggregates for 500 nM contamination study (all volumes in μL)

| | HEPES buffer | Liposomes (1.4 | MgSO₄ | Heparin (1 μM | Over-sulfated |
|---|---|---|---|---|---|
| | (25 mM, pH 8) | mM total lipid) | at 2M | concentration) | contaminant |
| Heparin only | 122 | 50 | 6 | 178 | — |
| 0.5 mol % | 114 | 50 | 6 | 177.11 | 8.9 (100 nM) |
| contamination | | | | | |
| 1.0 mol % | 122 | 50 | 6 | 176.2 | 1.78 (1 μM) |
| contamination | | | | | |
| 2.5 mol % | 122 | 50 | 6 | 173.6 | 4.45 (1 μM) |
| contamination | | | | | |
| 5.0 mol % | 122 | 50 | 6 | 169.1 | 8.9 (1 μM) |
| contamination | | | | | |
| 10.0 mol % | 122 | 50 | 6 | 160.2 | 17.8 (1 μM) |
| contamination | | | | | |
| 15.0 mol % | 122 | 50 | 6 | 151.3 | 26.7 (1 μM) |
| contamination | | | | | |
| 20.0 mol % | 122 | 50 | 6 | 142.4 | 35.6 (1 μM) |
| contamination | | | | | |
| 30.0 mol % | 122 | 50 | 6 | 124.6 | 53.4 (1 μM) |
| contamination | | | | | |

### Results and Discussion

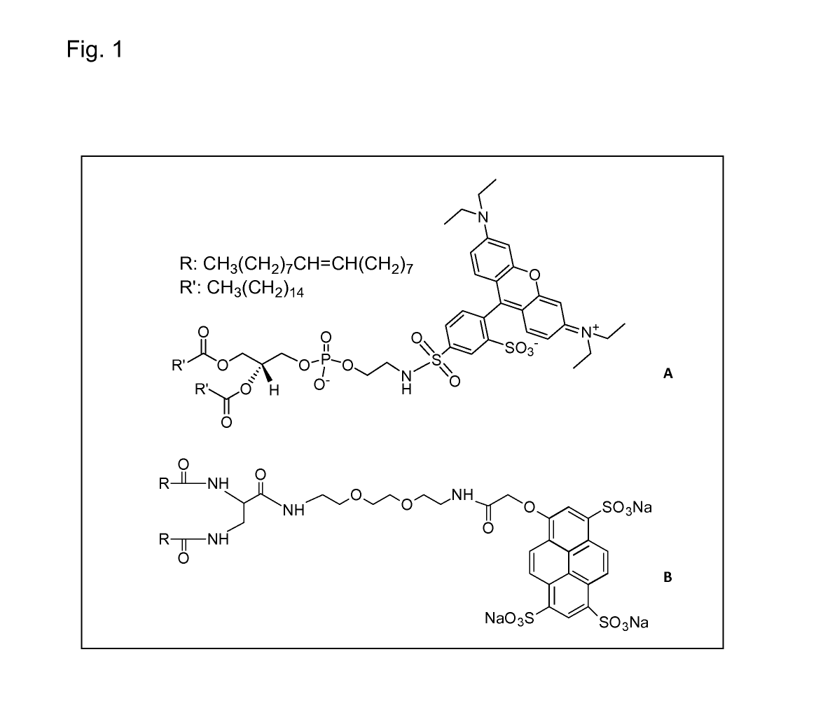

In our previous work, we have demonstrated that phosphocholine liposomes having either the pyranine lipid or the lissamine-rhodamine lipid present in the bilayer at 1 mol % were able to distinguish between different GAG species in solution²¹. In these studies, we have found the optimal liposomes for GAG discrimination contain the pyranine or the rhodamine lipid (FIG. 1 shows structures of these lipids); however preliminary studies demonstrated that liposomes containing the pyranine head group tend to aggregate in the presence of excess of divalent cations (i.e., in the absence of GAG; data not shown). Based on these prior results, we prepared 1-palmitoyl-2-oleoyl-sn-glycero-3-phosphocholine (POPC) liposomes incorporating 1 mol % of 1,2-dipalmitoyl-sn-glycero-3-phosphoethanolamine-N-(lissamine rhodamine B sulfonyl; rhodamine lipid), as well as 1,2-distearoyl-sn-glycero-3-phosphocholine (DSPC) liposomes incorporating 1 mol % rhodamine lipid for use in these studies. We employed transmission electron microscopy (TEM) and dynamic light scattering (DLS) to evaluate the relative diameter differences between aggregates produced by different GAGs. Changes in diameter and zeta potential in the presence of different GAGs were also evaluated. We used DLS and zeta potential changes to determine if there are any variations upon contamination of heparin with over-sulfated GAGs. Inclusion of the fluorophore in the liposomal bilayer was originally intended for study of fluctuations in fluorescence emission intensity as a function of the aggregation phenomenon. However, due to non-uniformity of the liposomal solution upon aggregation, fluorescence studies produced very variable results, and were thus removed from this study.

Preparation of liposomes: We have previously shown that 100 nm diameter liposomes composed of 99 mol % POPC and 1 mol % fluorophore-conjugated lipid (either pyranine, rhodamine, or dansyl) are able to discriminate between various GAGs²¹. Although these liposomes undergo modulations in fluorescence intensity in the presence of GAGs only, we wish to utilize the tendency of these liposomes to undergo rapid changes in the aggregate diameter and zeta potential in the presence of GAG and divalent cations to develop a rapid screen for these contaminants To achieve this, we have chosen Mg²⁺ as a flocculating agent²², and have produced POPC liposomes of three diameters (50, 200, and 550 nm) and aggregated each of these in the presence of three concentrations (50, 170, and 500 nM) of each GAG of interest: heparin, over-sulfated heparin (OSH), over-sulfated chondroitin sulfate (OSCS), and over-sulfated dermatan sulfate (OSD). We demonstrate that high concentrations of Mg²⁺ aggregate liposomes in the presence of GAG, but not in the absence of GAG (as shown in Tables 5 and 6).

Mechanistic studies—liposomes selectively aggregate upon binding of different GAG species when Mg²⁺ is present, and liposome-GAG interactions influence the zeta potentials and diameters of overall assembly: Kim and Nishida had proposed that the divalent cation (Mg²⁺ in our case) form bridges between the negative phosphate groups of the phospholipid head groups⁵. This interaction shields the incoming GAGs from the negative charges on liposome surface, allowing them to bind to the positively charged choline⁵, leading to the formation of aggregates.

To study this effect, we used both DSPC-rhodamine liposomes and POPC-rhodamine liposomes (200 nm diameter) in the presence of Mg²⁺ only, in the presence of each GAG only (no Mg²⁺), and finally in the presence of both GAG and Mg²⁺ (GAG concentration was held constant at 170 nM). Both diameters and zeta potentials of the resulting aggregates were measured. Results of these studies are as shown in Tables 5 and 6.

TABLE 5
Diameters and zeta potentials of POPC-containing liposomes
in the presence of GAG, with and without Mg²⁺

| Formulation | Zeta potential (mV) | Diameter (nm) |
|---|---|---|
| Liposomes only | −13.3 ± 0.78 | 183.1 ± 8.01 |
| Liposomes + Heparin | −11.6 ± 1.16 | 177.0 ± 3.94 |
| Liposomes + OSH | −12.1 ± 0.80 | 174.4 ± 6.35 |
| Liposomes + OSCS | −12.3 ± 0.37 | 186.8 ± 3.2 |
| Liposomes + OSD | −12.2 ± 0.41 | 173.5 ± 4.45 |
| Liposomes + Mg²⁺ | 4.4 ± 0.61 | 179.5 ± 1.58 |
| Liposomes + Mg²⁺ + Heparin | 4.6 ± 0.58 | 540.8 ± 50.49 |
| Liposomes + Mg²⁺ + OSH | 4.7 ± 0.75 | 773.9 ± 78.54 |
| Liposomes + Mg²⁺ + OSCS | 3.2 ± 0.97 | 2098.6 ± 192.87 |
| Liposomes + Mg²⁺ + OSD | 3.7 ± 0.80 | 3325.8 ± 543.79 |

TABLE 6
Diameters and zeta potentials of DSPC-containing liposomes
in the presence of GAG, with and without Mg²⁺.

| Formulation | Zeta potential (mV) | Diameter (mn) |
|---|---|---|
| Liposomes only | −11.8 ± 0.28 | 254.9 ± 90.97 |
| Liposomes + Heparin | −11.4 ± 0.81 | 374.2 ± 61.16 |
| Liposomes + OSH | −11.9 ± 0.44 | 445.9 ± 68.92 |
| Liposomes + OSCS | −11.7 ± 0.43 | 429.4 ± 36.72 |
| Liposomes + OSD | −12.7 ± 0.34 | 426.6 ± 58.89 |
| Liposomes + Mg²⁺ | 10.3 ± 0.91 | 397.7 ± 96.19 |
| Liposomes + Mg²⁺ + Heparin | 6.7 ± 1.11 | 2603 ± 189.51 |
| Liposomes + Mg²⁺ + OSH | 6.7 ± 1.08 | 1873 ± 162.66 |
| Liposomes + Mg²⁺ + OSCS | −16.0 ± 0.62 | 2483 ± 200.76 |
| Liposomes + Mg²⁺ + OSD | −2.7 ± 0.67 | 3489 ± 762.22 |

As the zeta potentials of both POPC and DSPC-containing liposomes do not appear to change significantly without the presence of Mg²⁺, we conclude that the GAGs alone do not bind to the surface of liposomes, as has been previously reported⁴. However in contrast to previous studies⁴, we find that excess Mg²⁺ does result in liposomal charge inversion²³, changing the zeta potential of the liposomes. It is likely that previous studies did not use divalent cations in sufficiently large excess to observe this effect. Consistent with previous findings, we note a significant change in the zeta potential upon the addition of both Mg²⁺ and GAG in the presence of DSPC liposomes; however this effect is negligible for POPC liposomes. Interesting to note is the drop in zeta potential of the DSPC aggregates to −16 mV in the presence of OSCS, 4 mV below that of the original liposomes. This effect likely results from overcharging of the liposome surface, due to excess charge from the OSCS²³. Both liposomes experience significant changes in aggregate diameter in the presence of GAG and Mg²⁺, and these diameter changes appear to be dependent on the species of GAG present, particularly for POPC liposomes. It must also be noted that previous studies by M. Krumbiegel and K. Arnold describe the measurement of zeta potential in the presence of liposomes aggregated by glycosaminoglycans, and they have found that this aggregation in no way interferes with the measurement of zeta potential².

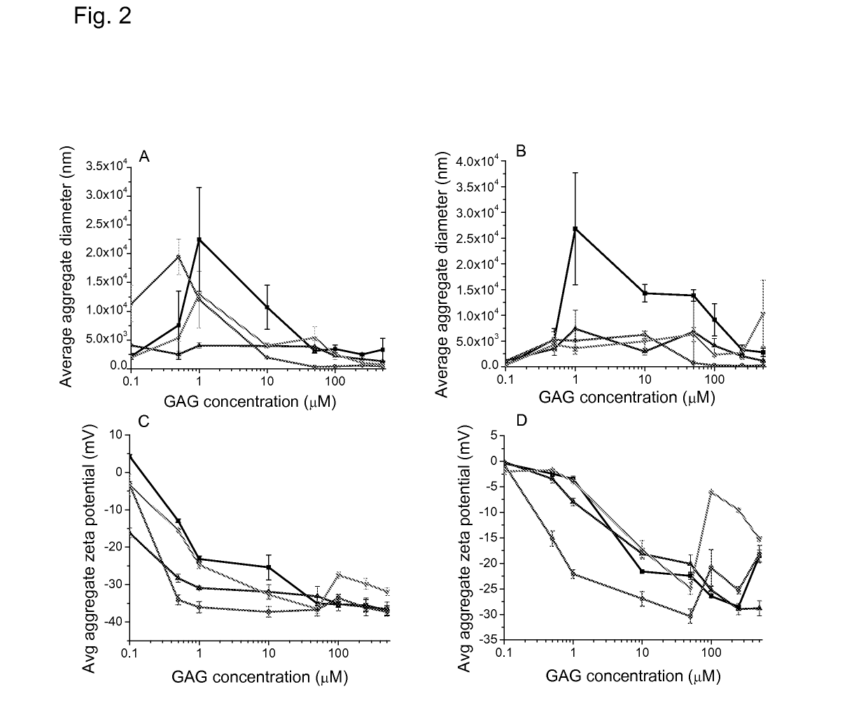

Mechanistic studies—diameter and zeta potential of liposome aggregates reach saturation upon addition of sufficient concentrations of GAG: To determine how the aggregate hydrodynamic diameters and zeta potentials of both DSPC and POPC containing liposomes changed with increasing concentrations of each GAG, and to determine if there were any differences between GAGs at these concentrations, DSPC and POPC liposomes were incubated with heparin, over-sulfated chondroitin sulfate, over-sulfated dermatan sulfate, and over-sulfated heparin at eight concentrations (100 nM, 500 nM, 1 μM, 10 μM, 50 μM, 100 μM, 250 μM, and 500 μM). Results are summarized in FIG. 2 below; each data point is the average of three collected aggregate diameters or zeta potential measurements. We note that some of the diameter measurements are outside the range which the Zetasizer Nano may accurately measure (5×10³ nm diameter), however the purpose of these experiments was to investigate whether each species of GAG caused the eventual saturation of both aggregate diameters and zeta potentials, and if so above what concentration does this saturation take place. Measurements are of interest in terms of general trend only. Notable are the progression of aggregate diameters from small to quite large and then to small again for both DSPC and POPC containing liposomes, as concentration increases. This is consistent with theoretical analysis of McClements²⁴ and Guzey²⁵, according to which below a specific critical concentration of charged polymers (e.g., GAGs), the surface of the colloid particles (liposomes) will be incompletely covered by the polymer, resulting in an imbalance between attractive and repulsive forces acting on the colloidal particles. Below this critical concentration, these imbalances will allow sections of liposome surface coated with GAG to attract sections of neighboring liposomes which have not been so coated, resulting in aggregate formation. Above this critical concentration however, the surfaces of the colloidal particles will become saturated as the charged polymer forms a continuous coat on the surface, and allows the repulsive forces between the colloid particles in solution to become re-balanced, preventing significant aggregation. McClements²⁴ also notes that at concentrations much higher than the critical concentration may cause “depletion flocculation” due to excesses of polymer electrolyte in solution, which may be sufficient to overcome the repulsive forces between colloid particles. This depletion flocculation may be one explanation for the sudden increase in diameter of the POPC liposomes in presence of 500 μM over-sulfated heparin.

For the DSPC containing liposomes, as the aggregate diameter becomes saturated, the zeta potential becomes likewise saturated (at high GAG concentration), and does not change appreciably at higher concentrations. For the POPC containing liposomes however, there is a tendency for the zeta potential to reach a minimum, and then return to smaller absolute values at higher concentrations. This difference is clearly due to the difference in composition of the fatty acid tails of the liposomal lipids. In the case of DSPC, both tails are constituted of saturated (stearic acid) and thus they will pack more efficiently (vis a vis the palmitoyl and oleyl tails of POPC) within the lipid phase. These differences will impart greater rigidity to the head groups of the DSPC liposomes, and thus will allow homogeneous distribution of GAG induced aggregates of the liposomes. The above feature is unlikely to prevail in the case of POPC liposomes. It is worth noting that other studies involving changes in the liposome's zeta potential upon addition of GAGs and divalent cations were focused on lipid bilayers, harboring saturated lipids (DMPC, DLPC, and egg lecithin)^{2,4}, and these studies produced zeta potential results similar to our DSPC liposomes. However, irrespective of the underlying physical forces responsible for our observed experimental data of FIG. 2, it is evident that POPC and DSPC formulated liposomes elicit marked differences in their aggregational states and zeta potentials as a function of different types of GAGs. Whether or not such features are intimately involved in discriminatory changes in the liposome's resident fluorescence probes²¹ as a function of different types of GAGs are currently being investigated in our laboratory, and we will report these findings subsequently.

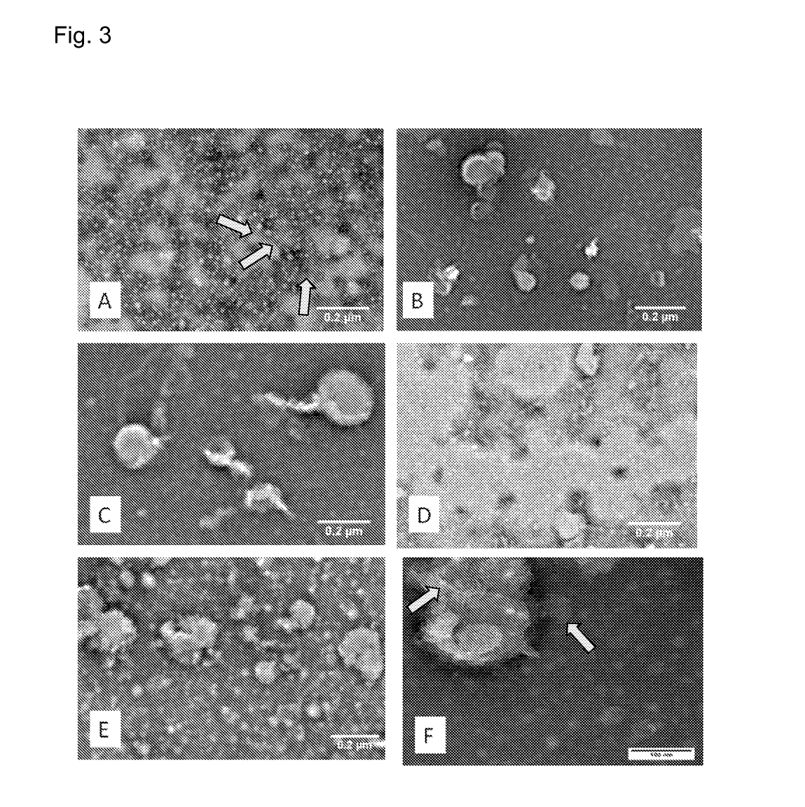

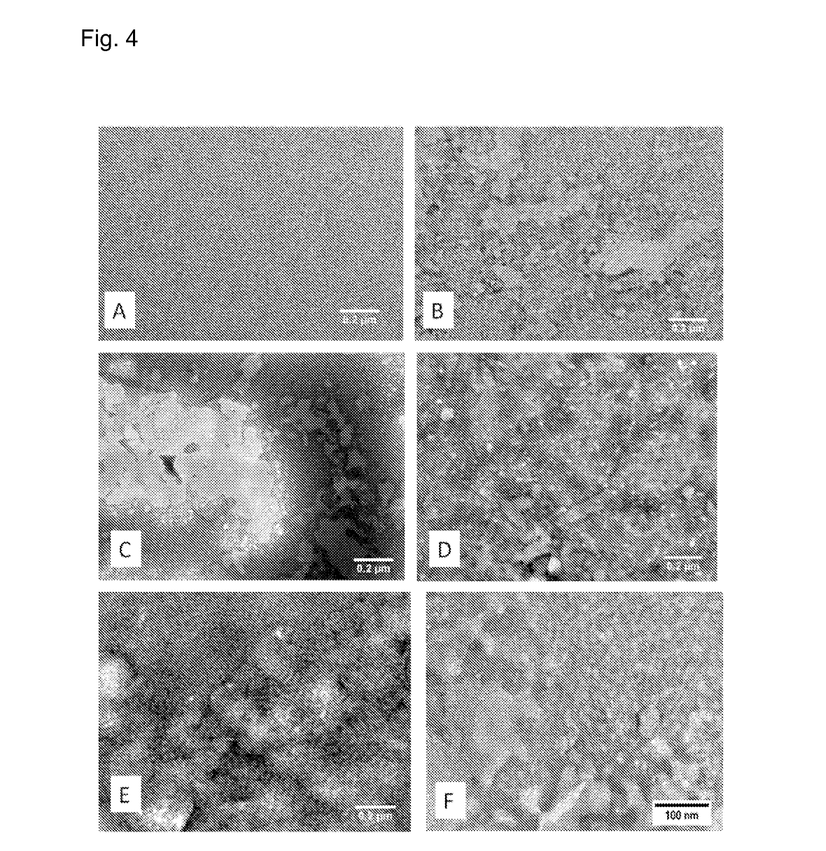

TEM images demonstrate differential aggregation of liposomes in the presence of different GAG species: The diameters of the POPC liposomes and DSPC liposomes in the presence of Mg²⁺ only were compared with those in the presence of heparin, over-sulfated chondroitin sulfate, over-sulfated dermatan sulfate, and over-sulfated heparin. FIG. 3 presents the TEM images of the POPC liposomes in the presence of Mg²⁺ alone (panel A) and in the presence of Mg²⁺ and different GAG species. FIG. 4 presents the corresponding TEM images involving DSPC liposomes. In each figure, panels A-E are images of liposomes magnified 5,000 times, and panel F is an image of one OSCS aggregate magnified 25,000 to show detail of the stacked liposomes. The TEM images of FIGS. 3 and 4 clearly reveal that the liposomes are aggregated in the presence of Mg²⁺ and different GAG species, and such aggregates are asymmetrical and polydisperse. However, notable in these TEM images is the presence of considerably larger aggregates in the presence of over-sulfated GAGs as compared to those observed in the presence of heparin. Also notable is the apparent size in these images; it is evident that the liposomes and aggregates have collapsed during the preparation of the samples. It is therefore necessary to consider these sizes as relative; aggregate images should only be compared with images of the liposomes in the presence of Mg²⁺ only.

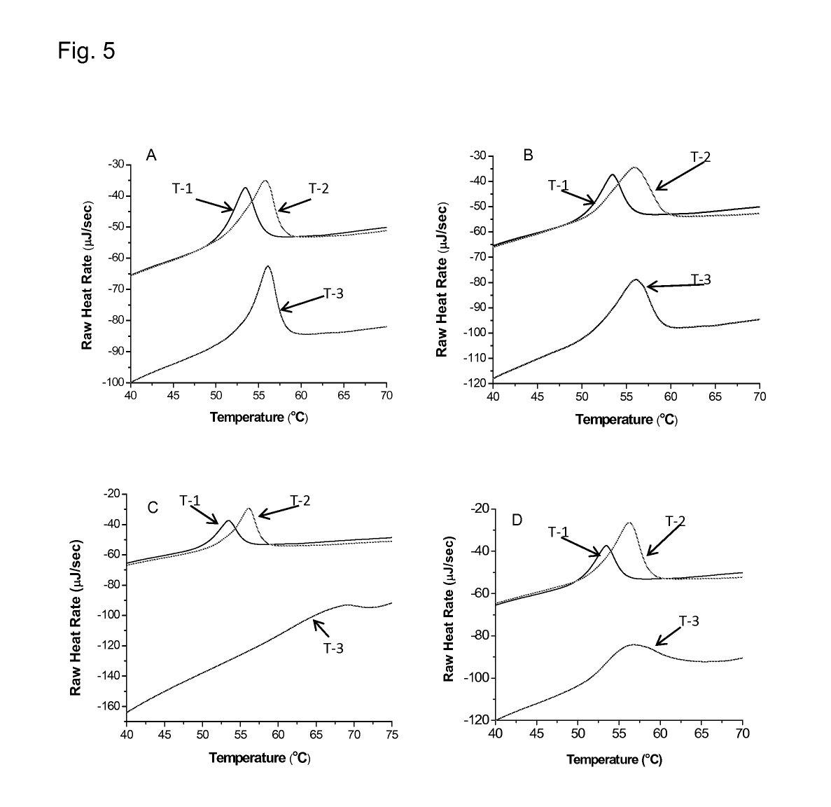

Differential scanning calorimetry demonstrates intrinsic and differential stability of liposomes upon binding of GAGs to liposomes: Having established that the liposomes are aggregated in the presence of both Mg2+ and different GAG species, it was of interest to investigate whether the above “effectors” modulated the intrinsic stability of liposomes. To probe this, we performed DSC studies for melting of DSPC liposomes in the presence of Mg2+ and two concentrations (i.e., 1 μM and at 250 μM) of each different GAG species. The DSC endotherms reveal that the presence of Mg2+ and GAGs influence both the melting temperature (Tm value) of the liposomes as well as the area under the peaks (measure of the enthalpic changes between native and denatured/melted forms of the liposomes; see FIG. 5). The observed shifting of the 250 μM trace to a lower relative heat rate reflects the increase of dissolved solutes over the control26, and the widening and flattening of the DSC trace with increasing GAG concentration, accompanied by a rightward shift in Tm, indicates that structural changes are taking place within the bilayers of the liposomes (increased Tm), and that these changes are dispersed somewhat unevenly within the “population” of the liposomes (widening and flattening of the Tm peak)26. To our further interest, we observed that the second DSC scan (performed after cooling the heated sample after the first scan) yielded essentially identical Tm values in the presence of different GAG species, albeit the enthalpic changes were slightly decreased (data not shown). This suggests that there is a marked reversibility in the organizational states of the liposomes, and such feature is intrinsic to the nature of the GAG species. Table 7 summarizes the Tm values and enthalpic changes under our selected experimental conditions. A perusal of the data of Table 7 reveals that among different GAGs used herein, heparin and oversulfated heparin exhibit the least and most stabilizing influence on the liposomes as evident by their corresponding enthalpic changes.

We conclude from these studies that binding of GAGs and Mg²⁺ to the liposomal bilayer causes the liposomal assembly to become more stable, and thus requires more heat energy (enthalpic changes) to bring it to the fully disorganized (melted) states with concomitant increase in the transition temperature. We believe the above feature is due to the intercalation of the GAGs between the individual phosphocholine molecules, thus forcing the exclusion of intervening water molecules and thus allowing the liposomal lipids to pack more efficiently in their native states.

TABLE 7
Heat required for liposome melting (μJ)

| GAG | Liposomes only | 1 μM | 250 μM |
|---|---|---|---|
| Heparin | 1709.1 | 2814.9 | 2887.1 |
| OSCS | 1709.1 | 3338.2 | 3367.5 |
| OSD | 1709.1 | 3693.2 | 2793.5 |
| OSH | 1709.1 | 3653.9 | 3853.4 |

Mechanistic studies: there is an inverse relationship between the percent change of aggregate diameter and the percent change of aggregate zeta potential as the concentration of GAG increases, independent of liposome diameter: For studies comparing the relative contribution of liposome diameter and GAG concentration to the overall average diameter and zeta potential changes of the resulting aggregates, only POPC liposomes were used. This is due to the high variability of the DSPC liposomes' diameters, which is clear from results shown in Table 6 (the standard deviation for the diameter of these liposomes alone as measure by DLS is 36% of their diameter). Additionally, DLS shows the presence of both very large (>1000 nm) and very small (<50 nm) particles in the DSPC liposome solution. Due to this difficulty in controlling the liposome diameter, DSPC liposomes have been excluded from this, as well as the contamination studies.

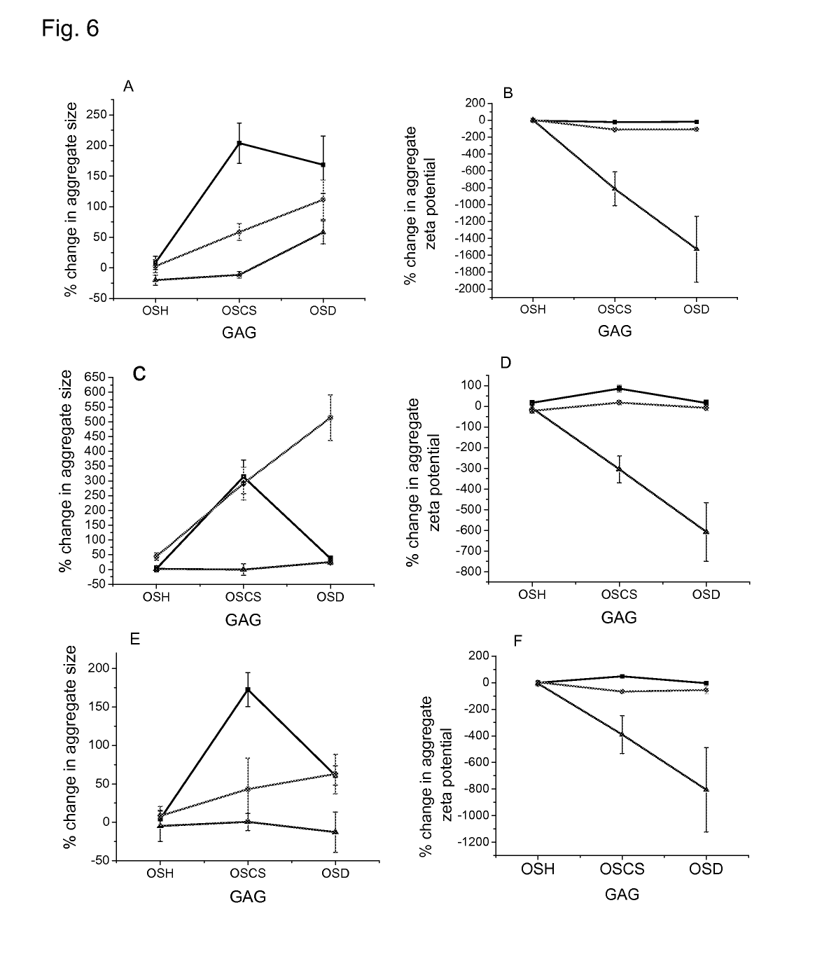

As one considers the percent change of each over-sulfated contaminant relative to heparin at each concentration, while holding the diameter of the liposomes constant, an interesting pattern emerges: there appears to be an inverse relationship between the percent change in aggregate diameters, and the percent change in aggregate zeta potential (i.e.—as the percent change in diameter goes down with increasing GAG concentration, the percent change in zeta potential becomes greater with increasing GAG concentration). These results are summarized in FIG. 6. Notable from this figure is that at 50 nM concentration (represented by a black trace with black squares), OSCS always produces the greatest change in aggregate diameter, regardless of the liposomes' starting diameter. At 170 nM GAG, OSD causes the greatest changes in aggregate diameter, and at 500 nM GAG results depend on the starting liposome diameter. Reasons for this are unclear, and will require further investigation. However it is obvious from these results that as GAG concentration increases, overall percent change decreases. Results for percent change in aggregate zeta potential are also very consistent for liposomes of all diameters tested: as GAG concentration increases, the magnitude of percent change in aggregate zeta potential also increases. We hypothesize the mechanism for this may be due to differences in the percent overall coverage of the liposome surface by the GAG. When the concentration of GAG in solution is relatively low relative to the total lipid concentration in solution (˜200 nM), the liposomal surface is covered with GAG to a lesser extent, resulting in greater imbalance between the attractive and repulsive colloidal forces. As such, the number of liposomes which form aggregates will be dependent on the charge density of the GAG present on the liposome surface, as well as the surface area between oppositely charged sections of each bilayer (a function both of liposome diameters and the percent of surface area covered). However, as the concentration of GAG in solution increases, the surface of each liposome bilayer will be covered to a greater extent, which will not only begin to re-balance the repulsive forces between them in solution, but it will also reduce the amount of available surface area for aggregation between liposomes. This will reduce the percent change in the aggregate diameter (as fewer liposomes will be able to aggregate together), as well as increasing the change observed in the zeta potentials (as a function of the amount and charge density of the GAG bound). Studies to confirm this mechanism are currently being undertaken.

Contamination studies demonstrate that changes in diameter and zeta potential of POPC liposomes can distinguish small changes in GAG composition: The insights gained from the previous studies were employed to probe whether the presence of low concentrations of over-sulfated contaminants in a heparin sample could be detected using DLS and zeta potential measurements of liposomal aggregates. We chose to incubate 200 nm diameter liposomes with 170 nM contaminated heparin (produced the greatest percent changes in diameter), and 500 nm diameter liposomes with 500 nM contaminated heparin (produced the greatest percent changes in zeta potential). Heparin samples in 2008 were found by Beyer, et al, to be contaminated in the range of 0.5% to 28% by weight⁹. As such, for both of these liposome/GAG concentration combinations, eight contamination levels were prepared: 0.5, 1.0, 2.5, 5, 10, 15, 20, and 30 mol % contaminations with each over-sulfated contaminant Each combination was measured for changes in aggregate diameter and zeta potential by DLS.

Analysis of variance (α=0.05) was conducted for each of these sets of data (see statistical results in Supplementary Information). Included in this analysis is a comparison of means for each contamination level against heparin alone using Dunnett's method for pairwise comparisons²⁷. This method allows us to compare each contamination level to the control (heparin only) while controlling the family-wise error of all comparisons together to 0.05. Results for both 200 nm and 500 nm diameter liposomes indicate that OSH could not be consistently detected, and thus will be eliminated from further discussion.

Results for OSCS and OSD are far more promising. Analysis of variance indicates that for the 200 nm liposomes, changes in average aggregate diameter could detect contamination by OSCS at concentrations from 5 mol % to 30 mol %, and OSD contamination from concentrations of 10 mol % to 30 mol %. Changes in aggregate zeta potential could not consistently detect contamination. Results for the 500 nm diameter liposomes indicate detection of OSCS contamination at concentrations from 1 mol % through 30 mol % by changes in zeta potential, and from 2.5 mol % to 30 mol % by changes in aggregate diameter. OSD could be detected by this method from 10 mol % to 30 mol % by changes in zeta potential, and from 0.5 mol % to 30 mol % by changes in aggregate diameter. (For detailed statistical results please see Supplementary Information). If we consider percent heparin contamination by weight, the lowest contamination level we can detect using these methods is approximately 1.6% by weight of OSD, and 2.2% by weight of OSCS, making it an attractive screening tool for heparin intended for clinical use. These calculations are based on the estimated molecular weights of heparin, over-sulfated chondroitin sulfate, and over-sulfated dermatan sulfate, summarized in Table 8.

TABLE 8
Molecular weight of GAGs (g/mol)

| GAG | Liposomes only |
|---|---|
| Heparin | 13,500 |
| OSCS | 29,560 |
| OSD | 42,529 |

It must be stated that despite the relative consistency and significance of the DLS diameter measurements, the presence of fluorescence, high polydispersity, and large precipitating particles in the sample lead us to favor the use of zeta potential for measurements of over-sulfated heparin contaminants, as these measurements are unaffected by any of the aforementioned concerns.

A comparison of current methods used to detect heparin quality reported in 2011 by Alban, et al., has been very revealing. The authors reported that while NMR and other spectroscopic methods are useable, other heparin mimetic may cause deviating results, and thus accurate detection of OSCS in heparin will be in large part dependent on the skill of the individual running the tests, and only currently known heparin contaminants may be recognized¹⁸. Additionally, the PT and aPTT, while they are able to detect overall heparin quality, cannot actually detect contamination, and have an LOD of 3%¹⁸. Further, it must be recognized that no reported adverse effects were observed from enexoparin contaminated with up to 7% OSCS¹⁵. Based on this, the analysis by Alban and Beyer of original contaminated samples^{9,18}, and the above statistical analysis of our data, we believe that zeta potential measurements combined with DLS diameter measurements of POPC based liposomes incubated with heparin samples at 170/500 nM and excess Mg²⁺ may be a rapid and economical initial screen for contamination in these samples.

### Conclusions

We have demonstrated that liposomes containing 1 mol % lissamine-rhodamine lipid fonn aggregates of varying diameters and zeta potentials depending on the species and concentration of GAG present. This has been verified by TEM studies. We have shown that organizational states of the liposome bilayers are influenced by the presence of GAG and excess Mg²⁺, resulting in a stabilizing effect, and the magnitude of this effect is also dependent on GAG species and concentration present. Additionally, there is an inverse relationship between the percent change of aggregate diameter and percent change of aggregate zeta potential, as a function of GAG concentration in solution. Finally, the presence of small concentrations of over-sulfated contaminants in heparin samples cause statistically significant variations in the average aggregate diameter and zeta potential POPC liposomes. Significant variations of POPC liposome aggregate zeta potentials enables detection of over-sulfated chondroitin sulfate and over-sulfated dermatan sulfate at 1 mol % and 0.5 mol % (2.2% w/w and 1.6% w/w, respectively). Based on the work of Bayer, the use of this method would have been able to detect the contaminants in the majority of the original heparin samples which caused allergic reactions and deaths of patients in 2007 and 2008⁹. These results offer insight into the potential of these interactions for a rapid and economical screen for the presence of over-sulfated contaminants in heparin or other drugs.

### REFERENCES

(1) Zhang, F.; Zhang, Z.; Linhardt, R. J. The Handbook of Glycomics; Elsevier: London, UK, 2009.
(2) Krumbiegel, M.; Arnold, K. Chemistry and Physics of Lipids 1990, 54, 1-7.
(3) Satoh, A.; Toida, T.; Yoshida, K.; Kojima, K.; Matsumoto, I. FEBS Letters 2000, 477, 249-252.
(4) Zschornig, O.; Richter, W.; Paasche, G.; Arnold, K. Colloid Polymer Science 2000, 278, 637-646.
(5) Kim, Y. C.; Nishida, T. J Biol Chem 1977, 252, 1243-1249.
(6) Voet, D.; Voet, J. Biochemistry; 3rd ed.; John Wiley & Sons, Inc.: Hoboken, N.J., 2004.
(7) Linhardt, R. J. Journal of Medicinal Chemistry 2003, 46, 2551-2564.
(8) Maruyama, T.; Toida, T.; Imanari, T.; Yu, G.; Linhardt, R. Carbohydrate Research 1998, 306, 35-43.
(9) Beyer, T.; Matz, M.; Brinz, D.; Radler, 0.; Wolf, B.; Norwig, J.; Baumann, K.; Alban, S.; Holzgrabe, U. Eur J Pharm Sci 2010, 40, 297-304.
(10) Pan, J.; Qian, Y.; Zhou, X.; Pazandak, A.; Frazier, S. B.; Weiser, P.; Lu, H.; Zhang, L. Nature Biotechnology 2010, 28, 203-207.
(11) Li, B.; Suwan, J.; Martin, J. G.; Zhang, F.; Zhang, Z.; Hoppensteadt, D.; Clark, M.; Fareed, J.; Linhardt, R. J. Biochemical Pharmacology 2009, 78, 292-300.
(12) Pan, J.; Qian, Y.; Zhou, X.; Lu, H.; Ramacciotti, E.; Zhang, L. Journal of Biological Chemistry 2010, 285, 22966-22974.
(13) Zhang, Z.; Li, B.; Suwan, J.; Zhang, F.; Z., W.; Liu, H.; Mulloy, B.; Linhardt, R. Journal of Pharmaceutical Sciences 2009, 98, 4017-4026.
(14) Kang, Y.; Gwon, K.; Shin, J. H.; Nam, H.; Meyerhoff, M.; Cha, G. Analytical Chemistry 2011, 83, 3957-3962.
(15) Bairstow, S.; McKee, J.; Nordhaus, M.; Johnson, R. Analytical Chemistry 2009, 288, 317-321.
(16) Wang, L.; Buchanan, S.; Meyerhoff, M. Analytical Chemistry 2008, 80, 9845-9847.
(17) Sommers, C.; Mans, D.; Mecker, L.; Keire, D. Analytical Chemistry 2011, 8, 3422-3420.
(18) Alban, S.; Luhn, S.; Schiemann, S.; Beyer, T.; Norwig, J.; Schilling, C.; Radler, O.; Wolf, B.; Matz, M.; Baumann, K.; Holzgrabe, U. Anal Bioanal Chem 2011, 399, 605-620.
(19) Bertozzi, C. R.; Freeze, H. H.; Varki, A.; Esko, J. D. Glycans in biotechnology and the pharmaceutical industry; Cold Spring Harbor Laboratory Press: Cold Spring Harbor, N.Y., 2009.
(20) Nagasawa, K.; Uchiyama, H.; Wajima, N. Carbohydrate Research 1986, 158, 183-190.
(21) Nyren-Erickson, E. K.; Haldar, M. K.; Gu, Y.; Qian, S. Y.; Friesner, D. L.; Mallik, S. Analytical Chemistry 2011, 83, 5989-5995.
(22) Semerjian, L. A., G. M. Advances in Environmental Research 2003, 7, 389-403.
(23) Hsiao, P. Y. J Phys Chem B 2008, 112, 7347-7350.
(24) McClements, D. J. Langmuir 2005, 21, 9777-9785.
(25) Guzey, D.; McClements, D. J. Adv Colloid Interface Sci 2006, 128-130, 227-248.
(26) Gabbott, P. Principles and applications of thermal analysis; Blackwell Pub.: Oxford; Ames, Iowa, 2008.
(27) Mendenhall, W.; Sincich, T. A second course in statistics: regression analysis; 7th ed.; Pearson Education: Boston, 2012.

### Supplementary Information

### Example of Calculation of Total Lipid Concentration

MW of DSPC=790.145 g/mol, MW of Rhodamine lipid=1,249.641 g/mol

### Concentration of Total Lipid:

4.0×10⁻³ g DSPC×1 mol/760.076 g=5.26×10⁻⁶ mol
6.5×10⁻⁵ g rhodamine lipid×1 mol/1,249.641 g=5.2×10⁻⁸ mol
5.26×10⁻⁶ mol+5.2×10⁻⁸=5.312×10⁻⁶ mol
5.312×10⁻⁶ mol/3.8 mL=1.4×10⁻⁶ mol/mL
1.4×10⁻⁶ mol/mL×1000 mL/L=1.4×10⁻³ mol/L
=1.4 mM

### Statistical Analysis

All statistical analysis was carried out using Minitab (version 16.1.1, State College, Pa.). Raw data from the Zetasizer Nano (Malvern, Westborough, Mass.), including measurements of average diameter and zeta potential, were entered into the Minitab spreadsheets, and analysis was carried out using these numbers in their original form.

### Minitab Spreadsheets

One-Way ANOVA: 200 nm Liposomes OSCS Size Versus Contamination:

| Source | DF | SS | MS | F | P |
|---|---|---|---|---|---|
| contamination | 8 | 11431736 | 1428967 | 103.26 | 0.000 |
| Error | 36 | 498172 | 13838 | | |
| Total | 44 | 11929908 | | | |

| | | |
|---|---|---|
| S = 117.6 | R-Sq = 95.82% | R-Sq(adj) = 94.90% |

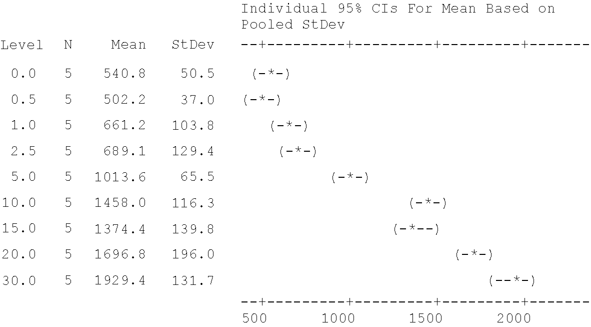

Pooled StDev = 117.6
Grouping Information Using Dunnett Method

| Level | N | Mean | Grouping |
|---|---|---|---|
| 0.0 (control) | 5 | 540.8 | A |
| 30.0 | 5 | 1929.4 | |
| 20.0 | 5 | 1696.8 | |
| 10.0 | 5 | 1458.0 | |
| 15.0 | 5 | 1374.4 | |
| 5.0 | 5 | 1013.6 | |
| 2.5 | 5 | 689.1 | A |
| 1.0 | 5 | 661.2 | A |
| 0.5 | 5 | 502.2 | A |

Means not labeled with letter A are significantly different from
control level mean.
Dunnett's comparisons with a control
Family error rate = 0.05
Individual error rate = 0.0084
Critical value = 2.79
Control = level (0) of contamination
Intervals for treatment mean minus control mean

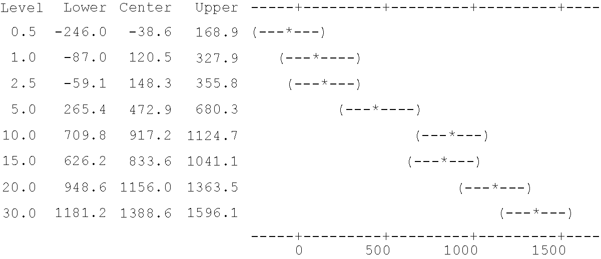

One-Way ANOVA: 200 nm Liposome OSD Size Versus Contamination:

| Source | DF | SS | MS | F | P |
|---|---|---|---|---|---|
| contamination | 8 | 4679965 | 584996 | 59.78 | 0.000 |
| Error | 36 | 352295 | 9786 | | |
| Total | 44 | 5032260 | | | |

| | | |
|---|---|---|
| S = 98.92 | R-Sq = 93.00% | R-Sq(adj) = 91.44% |

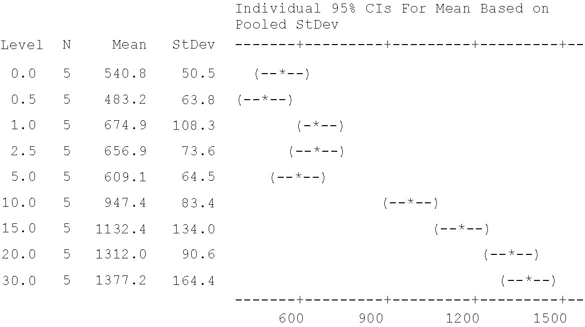

Pooled StDev = 98.9
Grouping Information Using Dunnett Method

| Level | N | Mean | Grouping |
|---|---|---|---|
| 0.0 (control) | 5 | 540.8 | A |
| 30.0 | 5 | 1377.2 | |
| 20.0 | 5 | 1312.0 | |
| 15.0 | 5 | 1132.4 | |
| 10.0 | 5 | 947.4 | |
| 1.0 | 5 | 674.9 | A |
| 2.5 | 5 | 656.9 | A |
| 5.0 | 5 | 609.1 | A |
| 0.5 | 5 | 483.2 | A |

Means not labeled with letter A are significantly different from
control level mean.
Dunnett's comparisons with a control
Family error rate = 0.05
Individual error rate = 0.0084
Critical value = 2.79
Control = level (0) of contamination
Intervals for treatment mean minus control mean

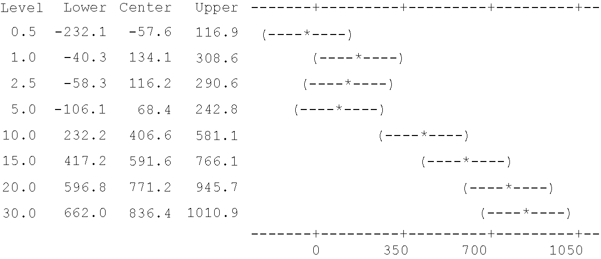

One-Way ANOVA: 200 nm Liposome OSCS Zeta Versus Contamination:

| Source | DF | SS | MS | F | P |
|---|---|---|---|---|---|
| contamination | 8 | 34.117 | 4.265 | 20.28 | 0.000 |
| Error | 36 | 7.571 | 0.210 | | |
| Total | 44 | 41.688 | | | |

| | | |
|---|---|---|
| S = 0.4586 | R-Sq = 81.84% | R-Sq(adj) = 77.80% |

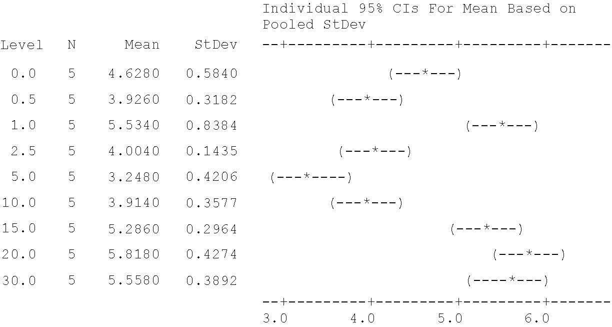

Pooled StDev = 0.4586
Grouping Information Using Dunnett Method

| Level | N | Mean | Grouping |
|---|---|---|---|
| 0.0 (control) | 5 | 4.6280 | A |
| 20.0 | 5 | 5.8180 | |
| 30.0 | 5 | 5.5580 | |
| 1.0 | 5 | 5.5340 | |
| 15.0 | 5 | 5.2860 | A |
| 2.5 | 5 | 4.0040 | A |
| 0.5 | 5 | 3.9260 | A |
| 10.0 | 5 | 3.9140 | A |
| 5.0 | 5 | 3.2480 | |

Means not labeled with letter A are significantly different from
control level mean.
Dunnett's comparisons with a control
Family error rate = 0.05
Individual error rate = 0.0084
Critical value = 2.79
Control = level (0) of contamination
Intervals for treatment mean minus control mean

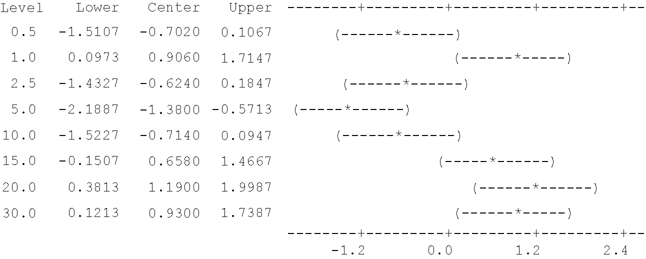

One-Way ANOVA: 200 nm Liposomes OSD Zeta Versus Contamination:

| Source | DF | SS | MS | F | P |
|---|---|---|---|---|---|
| contamination | 8 | 41.03 | 5.13 | 3.12 | 0.009 |
| Error | 36 | 59.23 | 1.65 | | |
| Total | 44 | 100.26 | | | |

| | | |
|---|---|---|
| S = 1.283 | R-Sq = 40.92% | R-Sq(adj) = 27.79% |

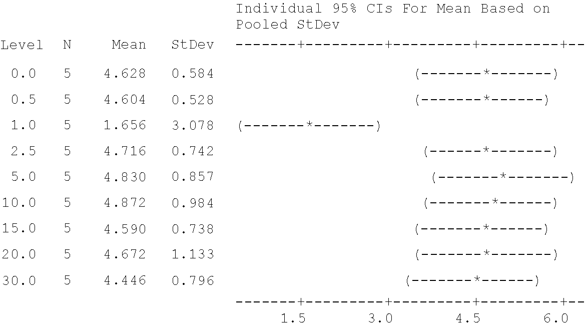

Pooled StDev = 1.283
Grouping Information Using Dunnett Method

| Level | N | Mean | Grouping |
|---|---|---|---|
| 0.0 (control) | 5 | 4.628 | A |
| 10.0 | 5 | 4.872 | A |
| 5.0 | 5 | 4.830 | A |
| 2.5 | 5 | 4.716 | A |
| 20.0 | 5 | 4.672 | A |
| 0.5 | 5 | 4.604 | A |
| 15.0 | 5 | 4.590 | A |
| 30.0 | 5 | 4.446 | A |
| 1.0 | 5 | 1.656 | |

Means not labeled with letter A are significantly different from
control level mean.
Dunnett's comparisons with a control
Family error rate = 0.05
Individual error rate = 0.0084
Critical value = 2.79
Control = level (0) of contamination
Intervals for treatment mean minus control mean

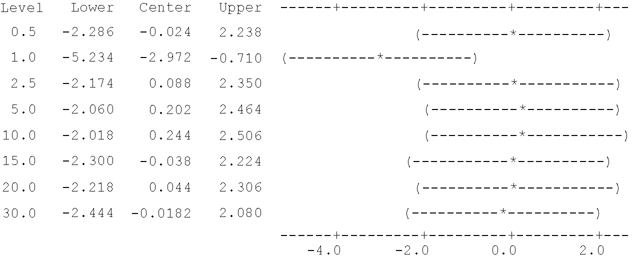

One-Way ANOVA: OSCS Size Versus Contamination:

| Source | DF | SS | MS | F | P |
|---|---|---|---|---|---|
| contamination | 8 | 4087429 | 510929 | 4.57 | 0.001 |
| Error | 36 | 4026851 | 111857 | | |
| Total | 44 | 8114280 | | | |

| | | |
|---|---|---|
| S = 334.5 | R-Sq = 50.37% | R-Sq(adj) = 39.35% |

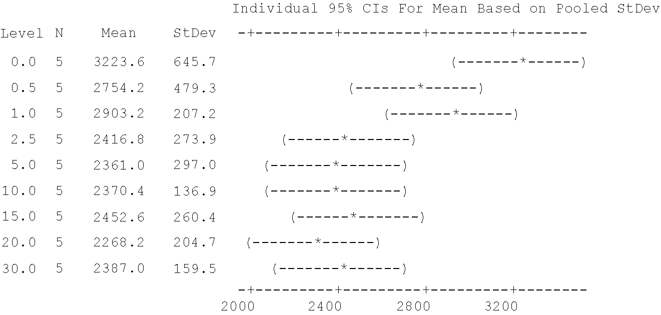

Pooled StDev = 334.5
Grouping Information Using Dunnett Method

| Level | N | Mean | Grouping |
|---|---|---|---|
| 0.0 (control) | 5 | 3223.6 | A |
| 1.0 | 5 | 2903.2 | A |
| 0.5 | 5 | 2754.2 | A |
| 15.0 | 5 | 2452.6 | |
| 2.5 | 5 | 2416.8 | |
| 30.0 | 5 | 2387.0 | |
| 10.0 | 5 | 2370.4 | |
| 5.0 | 5 | 2361.0 | |
| 20.0 | 5 | 2268.2 | |

Means not labeled with letter A are significantly different from
control level mean.
Dunnett's comparisons with a control
Family error rate = 0.05
Individual error rate = 0.0084
Critical value = 2.79
Control = level (0) of contamination
Intervals for treatment mean minus control mean

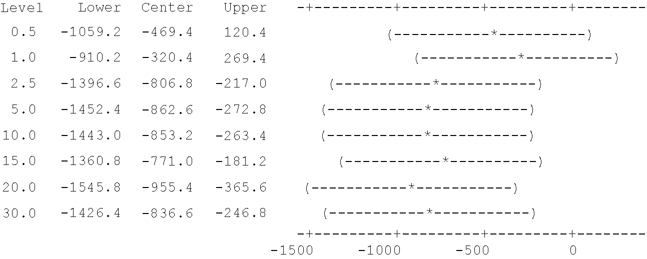

One-Way ANOVA: OSD Size Versus Contamination:

| Source | DF | SS | MS | F | P |
|---|---|---|---|---|---|
| contamination | 8 | 3786045 | 473256 | 5.67 | 0.000 |
| Error | 36 | 3005050 | 83474 | | |
| Total | 44 | 6791094 | | | |

| | | |
|---|---|---|
| S = 288.9 | R-Sq = 55.75% | R-Sq(adj) = 45.92% |

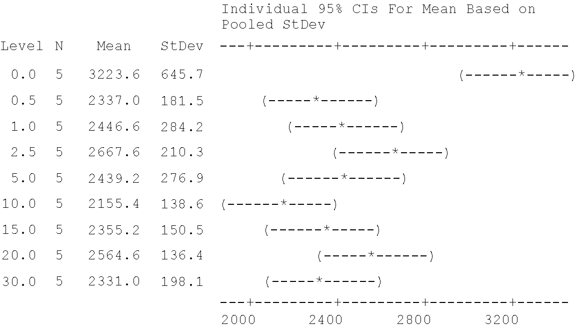

Pooled StDev = 288.9
Grouping Information Using Dunnett Method

| Level | N | Mean | Grouping |
|---|---|---|---|
| 0.0 (control) | 5 | 3223.6 | A |
| 2.5 | 5 | 2667.6 | |
| 20.0 | 5 | 2564.6 | |
| 1.0 | 5 | 2446.6 | |
| 5.0 | 5 | 2439.2 | |
| 15.0 | 5 | 2355.2 | |
| 0.5 | 5 | 2337.0 | |
| 30.0 | 5 | 2331.0 | |
| 10.0 | 5 | 2155.4 | |

Means not labeled with letter A are significantly different from
control level mean.
Dunnett's comparisons with a control
Family error rate = 0.05
Individual error rate = 0.0084
Critical value = 2.79
Control = level (0) of contamination
Intervals for treatment mean minus control mean

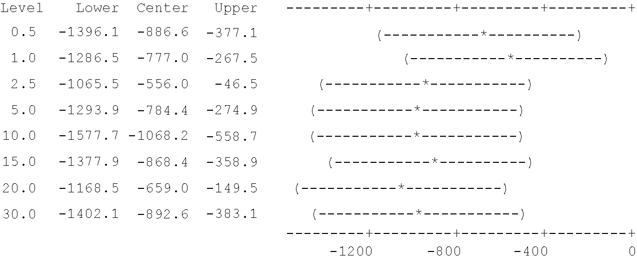

One-Way ANOVA: OSCS Zeta Versus Contamination:

| Source | DF | SS | MS | F | P |
|---|---|---|---|---|---|
| contamination | 8 | 426.64 | 53.33 | 31.69 | 0.000 |
| Error | 36 | 60.59 | 1.68 | | |
| Total | 44 | 487.24 | | | |

| | | |
|---|---|---|
| S = 1.297 | R-Sq = 87.56% | R-Sq(adj) = 84.80% |

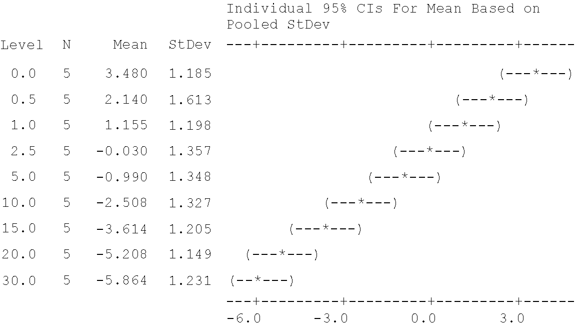

Pooled StDev = 1.297
Grouping Information Using Dunnett Method

| Level | N | Mean | Grouping |
|---|---|---|---|
| 0.0 (control) | 5 | 3.480 | A |
| 0.5 | 5 | 2.140 | A |
| 1.0 | 5 | 1.155 | |
| 2.5 | 5 | −0.030 | |
| 5.0 | 5 | −0.990 | |
| 10.0 | 5 | −2.508 | |
| 15.0 | 5 | −3.614 | |
| 20.0 | 5 | −5.208 | |
| 30.0 | 5 | −5.864 | |

Means not labeled with letter A are significantly different from
control level mean.
Dunnett's comparisons with a control
Family error rate = 0.05
Individual error rate = 0.0084
Critical value = 2.79
Control = level (0) of contamination
Intervals for treatment mean minus control mean

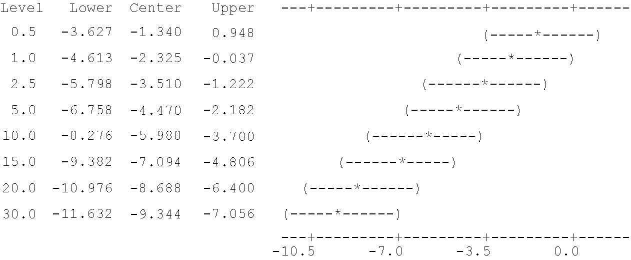

One-Way ANOVA: OSD Zeta Versus Contamination:

| Source | DF | SS | MS | F | P |
|---|---|---|---|---|---|
| contamination | 8 | 432.89 | 54.11 | 21.55 | 0.000 |
| Error | 36 | 90.40 | 2.51 | | |
| Total | 44 | 523.28 | | | |

| | | |
|---|---|---|
| S = 1.585 | R-Sq = 82.73% | R-Sq(adj) = 78.89% |

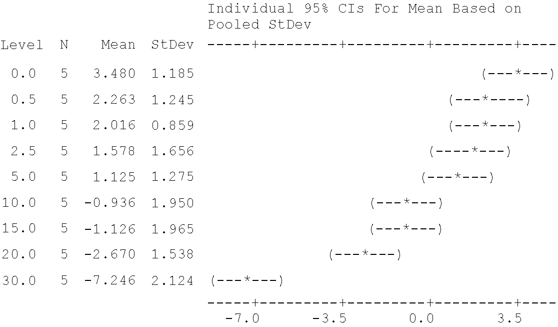

Pooled StDev = 1.585
Grouping Information Using Dunnett Method

| Level | N | Mean | Grouping |
|---|---|---|---|
| 0.0 (control) | 5 | 3.480 | A |
| 0.5 | 5 | 2.263 | A |
| 1.0 | 5 | 2.016 | A |
| 2.5 | 5 | 1.578 | A |
| 5.0 | 5 | 1.125 | A |
| 10.0 | 5 | −0.936 | |
| 15.0 | 5 | −1.126 | |
| 20.0 | 5 | −2.670 | |
| 30.0 | 5 | −7.246 | |

Means not labeled with letter A are significantly different from
control level mean.
Dunnett's comparisons with a control
Family error rate = 0.05
Individual error rate = 0.0084
Critical value = 2.79
Control = level (0) of contamination
Intervals for treatment mean minus control mean

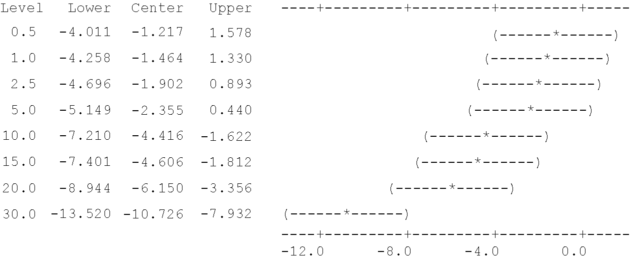

### Example 2

### Digestion of Heparin with Nitrous Acid Improves the Sensitivity of Liposomal Assay for Over-Sulfated Chondroitin Sulfate

In order to enhance the sensitivity of detecting charged contaminants in a test composition that includes heparin, while also making interpretation of results more user-friendly and eliminating the need for statistical analysis, heparin was digested with nitrous acid, prepared in situ by the mixing of hydrochloric acid (HCl) and sodium nitrite (NaNO₂). Nitrous acid is known to de-polymerize heparin, but not over-sulfated chondroitin sulfate (Zhang et al., 2008, J Med Chem 51:5498-5501). Following nitrous acid digestion, the low molecular weight heparin fragments had a significantly reduced effect on the size and zeta potential of the liposome aggregates. Any over-sulfated chondroitin sulfate present had a much greater effect relative to the heparin fragments, and was detectable in much lower amounts.

### Materials and Methods

Materials and synthesis of over-sulfated chondroitin sulfate (OSCS): All lipids used were obtained from Avanti Polar Lipids. Heparin and chondroitin-6-sulfate were obtained from Alfa Aesar and Spectrum Chemical Corp., respectively. Chondroitin was over-sulfated according to previously published procedures (Satoh et al., 2000, FEBS Letters 477:249-252; Maruyama et al., 1998, Carbohydrate Research 306:35-43).

Preparation of liposomes: Liposomes were prepared using 99 mol % POPC and 1 mol % rhodamine lipid using the technique described in Example 1. Briefly, lipids were dissolved in chloroform and mixed in a round-bottom flask at the appropriate ratios. Chloroform was flash evaporated at 50° C. using a rotary evaporator, forming a thin film of lipids on the inside of the flask. This thin film was dried under vacuum overnight to remove all traces of solvent. Four mL of 50 mM Tris buffer at pH 8 were then added to the thin film, and the flask was rotated at 50° C. for 20 minutes. The resulting liposomes were then extruded 15 times through a polycarbonate membrane filter of pore size 200 nm at 70° C. Final concentration of total lipid was calculated at 1.6 mM.

Heparin digestion experiments: For digestion with nitrous acid, solutions of heparin and over-sulfated chondroitin sulfate were prepared at two concentrations: 3 mg/mL and 10 mg/mL in deionized water. These solutions were combined with a solution of either sulfuric acid (H₂SO₄) or hydrochloric acid (HCl) at various concentrations, and sodium nitrite (NaNO₂) in water (dissolved just before use), also at various concentrations. The reaction was stopped by adding sodium hydroxide (NaOH) in water.

Table 9 below presents all combinations of acid, sodium nitrite, and base used. Digestion was allowed to proceed for 15, 30 and 60 minutes; NaOH was added after this incubation to stop the reaction. In all cases presented below, after digestion and addition of NaOH, liposomes were added to a final concentration of 200 μM total lipid, and MgSO₄ at 2 M concentration dissolved in water was added to a final concentration of 33 mM final concentration (approximate final volume for testing was 356 μL). The samples were allowed to incubate with the liposomes and MgSO₄ at room temperature for 15 minutes, 600 μL of 50 mM Tris buffer at pH 8 were added, and the samples were tested for aggregate diameter and zeta potential using a Malvern Zetasizer Nano ZS90. Each sample was read 3 times, using default settings.

TABLE 9
Heparin digestion combinations of acid, nitrite and base

| | | | | | Final Acid | Final Nitrite | Final Base |
|---|---|---|---|---|---|---|---|
| Method | Heparin/OSCS | Acid | Nitrite | NaOH | Concentration | Concentration | Concentration |
| A | 100 μL at | 20 μL | 2 μL at | 32.5 μL at | 0.08M | 0.16M | 0.1M |
| | 3 mg/mL | H₂SO₄ | 700 mg/mL | 0.5M | (0.16M H⁺) | | |
| | | at 0.5M | | | | | |
| B | 500 μL at | 20 μL | 2 μL at | 32.5 μL at | 0.02M | 0.04M | 0.03M |
| | 3 mg/mL | H₂SO₄ | 700 mg/mL | 0.5M | (0.04M H⁺) | | |
| | | at 0.5M | | | | | |
| C | 100 μL at | 20 μL | 2 μL at | 32.5 μL at | 0.08M | 0.16M | 0.1M |
| | 10 mg/mL | H₂SO₄ | 700 mg/mL | 0.5M | (0.16M H⁺) | | |
| | | at 0.5M | | | | | |
| D | 100 μL at | 20 μL | 2 μL at | 32.5 μL at | 0.16M | 0.16M | 0.1M |
| | 10 mg/mL | HCl at | 700 mg/mL | 0.5M | | | |
| | | 1.0M | | | | | |
| E | 100 μL at | 50 μL | 5 μL at | 81.25 μL at | 0.32M | 0.32M | 0.17M |
| | 10 mg/mL | HCl at | 700 mg/mL | 0.5M | | | |
| | | 1.0M | | | | | |
| F | 100 μL at | 50 μL | 14 μL at | 81.25 μL at | 0.3M | 0.92M | 0.165M |
| | 10 mg/mL | HCl at | 750 mg/mL | 0.5M | | | |
| | | 1.0M | | | | | |
| G | 100 μL at | 25 μL | 11 μL at | None | 0.92M | 0.87M | NA |
| | 10 mg/mL | HCl at | 750 mg/mL | Added | | | |
| | | 5.0M | | | | | |
| H | 100 μL at | 2 μL | 2 μL at | 3.0 μL at | 0.1M | 0.2M | 0.07M |
| | 3 mg/mL | HCl at | 750 mg/mL | 2.5M | | | |
| | | 5.0M | | | | | |
| I | 100 μL at | 8 μL | 8 μL at | 12 μL at | 0.4M | 0.74M | 0.23M |
| | 3 mg/mL | HCl at | 750 mg/mL | 2.5M | | | |
| | | 5.0M | | | | | |

Contamination experiments: To assess the sensitivity of the method to detect low amounts of OSCS in a sample of heparin, samples of contaminated heparin were produced at two concentrations: 3 mg/mL and 10 mg/mL. Table 10 below details the production of these contaminated samples. Heparin with no contamination was used as a control. Following mixing, the 3 mg/mL samples were digested using Method I above, the 10 mg/mL samples were digested using Method D above, each for 30 minutes (NaOH added only after the 30 minute incubation to stop digestion). Following digestion and addition of NaOH, liposomes were added to a final concentration of 200 μM total lipid, and MgSO₄ added to a final concentration of 33 mM, and these samples were incubated at room temperature for 15 minutes. Six hundred microliters (600 μL) of 50 mM Tris buffer at pH 8 were then added, and the samples were tested for aggregate diameter and/or zeta potential using the same equipment and settings described previously.

TABLE 10
Mixing of heparin samples with OSCS contamination

| Contam- | 3 mg/ml samples | | 10 mg/mL samples | |
|---|---|---|---|---|
| ination | Heparin | | Heparin | |
| % (w/w) | (3 mg/mL) | OSCS | (10 mg/mL) | OSCS |
| 0.05% | 100 μL | 2 μL | 100 μL | 2 μL |
| | | (0.075 mg/mL) | | (0.25 mg/mL) |
| 0.1% | 100 μL | 2 μL | 100 μL | 2 μL |
| | | (0.15 mg/mL) | | (0.5 mg/mL) |
| 0.5% | 100 μL | 2 μL | 100 μL | 1.7 μL |
| | | (0.75 mg/mL) | | (3 mg/mL) |
| 1.0% | 99 μL | 1.0 μL | 99 μL | 1.0 μL |
| | | (3 mg/mL) | | (10 mg/mL) |
| 5.0% | 95 μL | 5.0 μL | 95 μL | 5.0 μL |
| | | (3 mg/mL) | | (10 mg/mL) |
| 10.0% | 90 μL | 10.0 μL | 90 μL | 10.0 μL |
| | | (3 mg/mL) | | (10 mg/mL) |

### Results and Discussion

During the nitrous acid digestion of heparin, much shorter disaccharide fragments are created. We hypothesized that these shorter fragments will have a much reduced interaction with the surface of the liposomes, and thus will cause less variation in the resulting liposome aggregates' size and zeta potential. However, OSCS is not digested by nitrous acid, and thus will cause much greater variation in the aggregates' size and zeta potential as compared to digested heparin. When OSCS contaminates heparin at low levels, we hypothesized that nitrous acid digestion of the heparin will allow the contaminating OSCS to cause greater variations in the size or zeta potential of the aggregates formed. These variations will be much greater in magnitude after digestion than otherwise, allowing for more sensitive detection of the OSCS.

Heparin digestion trials: During the nitrous acid digestion procedures, the objective was to find the combination of heparin/OSCS, acid, nitrite, and base concentrations which would eventually lead to the largest difference between aggregates produced by heparin and OSCS. That is, following digestion we wish to produce liposome aggregates in the presence of heparin which are much different than those produced in the presence of OSCS, in size, zeta potential, or both. With these considerations, the two procedures selected for further study were methods ‘D’ and ‘I’ from Table 9 in the Materials and Methods section: method D yielded the greatest difference in aggregate zeta potentials, and method I yielded the greatest differences in aggregate sizes. Data from these studies is presented in Tables 11 and 12 below.

TABLE 11
Changes in liposome aggregate zeta potential in the presence
of heparin and OSCS following digestion by Method D.

| 15 min digest | |
|---|---|
| Liposomes only | 5.53 |
| Heparin | 2.05 |
| OSCS | −21.30 |
| 30 min digest | |
| Liposomes only | 6.26 |
| Heparin | −12.03 |
| OSCS | −22.00 |
| 60 min digest | |
| Liposomes only | 5.46 |
| Heparin | 1.55 |
| OSCS | −21.97 |

All numbers are an average of 3 runs.

Zeta potential is the electric potential at the boundary of hydrodynamic shear of a particle in solution (Malvern Instruments Ltd., Zeta Potential: An introduction in 30 minutes, available online at malvern.com). Thus, if negatively charged polymers (such as heparin or OSCS) adsorb to the surface of a positively charged particle (such as a liposome), the zeta potential will appear to become more negative (or less positive). As seen in Table 11 above, the liposomes have a positive zeta potential in the absence of heparin or OSCS. Following digestion, OSCS imparts a much more negative zeta potential to the liposome aggregates than heparin. Ideally, heparin in pure form would produce a slightly positive zeta potential, with addition of OSCS creating a negative zeta potential, as appears to be the case after digesting for 15 and 60 minutes. The 30 minute digest still produces a large spread between the zeta potentials of liposome aggregates in the presence of heparin and OSCS, but why the heparin produces a negative zeta potential in this case is unclear.

In Table 12 there are three important pieces of information: the Z-average diameter of the liposomes or liposome aggregates, the diameters of the distribution peaks (Pk) for each population detected, and the relative intensities of these distribution peaks. The Z-average diameter indicates the overall average of all aggregates from all size populations in solution. The presence of more than one distribution peak, Pk, up to 3, indicates the presence of more than one size population (Malvern Instruments Ltd., Dynamic Light Scattering: An introduction in 30 minutes, available online at malvem.com). For example, under the 15 minute heading, the liposomes only have a single peak with an indicated liposome diameter of 177.93 nm, indicating there is a single population of liposomes in solution with diameter 177.93 nm. However, the OSCS after 15 minutes of digestion produced aggregates of two size populations, one with a diameter of 876.37 nm and one with a diameter of 138.10 nm. Using this example again, the relative percent intensities of these populations are 58.47 and 41.53, indicating that the relative percent of scattering intensity is 58.47% from the larger aggregates and 41.53% from the smaller aggregates. From this it becomes clear that the presence of OSCS is forming larger aggregates than heparin after digestion.

TABLE 12
Changes in liposome aggregate diameter in the presence
of heparin and OSCS following digestion by Method I.

| | | | | | | |
|---|---|---|---|---|---|---|
| Z-average | | | | Pk 1 | Pk 2 | Pk 3 |
| diameter | Pk 1 | Pk 2 | Pk 3 | Area | Area | Area |
| (nm) | diameter* | diameter | diameter | % | % | % |

15 min digest

| | | | | | | | |
|---|---|---|---|---|---|---|---|
| Liposomes only | 170.33 | 177.93 | 0.00 | 0.00 | 100.00 | 0.00 | 0.00 |
| Heparin | 177.10 | 189.33 | 5052.67 | 0.00 | 98.53 | 1.47 | 0.00 |
| OSCS | 286.70 | 876.37 | 138.10 | 0.00 | 58.47 | 41.53 | 0.00 |

30 min digest

| | | | | | | | |
|---|---|---|---|---|---|---|---|
| Liposomes only | 157.27 | 184.47 | 0.00 | 0.00 | 100.00 | 0.00 | 0.00 |
| Heparin | 160.93 | 178.27 | 1749.00 | 0.00 | 99.63 | 0.37 | 0.00 |
| OSCS | 297.13 | 1245.33 | 122.70 | 1787.33 | 56.93 | 42.03 | 1.03 |

60 min digest

| | | | | | | | |
|---|---|---|---|---|---|---|---|
| Liposomes only | 154.23 | 173.27 | 1749.00 | 0.00 | 99.63 | 0.37 | 0.00 |
| Heparin | 153.93 | 170.83 | 1787.67 | 0.00 | 99.63 | 0.37 | 0.00 |
| OSCS | 216.03 | 146.70 | 1105.93 | 3418.33 | 67.50 | 30.93 | 1.60 |

*Diameters and areas are calculated by the DLS software based on the intensity of the scattered light. All numbers are an average of 3 runs.

Contamination experiments: As methods ‘D’ and T yielded the greatest differences in liposome aggregate zeta potential and size, respectively, between heparin and OSCS after digestion, these methods were used in the experiments using the contaminated heparin described in Table 10. Method ‘D’ makes use of the 10 mg/mL heparin samples, and method T makes use of the 3 mg/mL samples. Digestion using method D was allowed to incubate for 15 minutes at room temperature before stopping, and method I was allowed to incubate for 30 minutes, as these were the digestion times that resulted in the greatest difference between heparin and OSCS during optimization. Following digestion and analysis using the procedures outlined in the Materials and Methods section, the following results were obtained (Table 13).

TABLE 13
Results of contamination tests using methods ‘D’ and ‘I’

| | Method D | Method I | | | | | | |
|---|---|---|---|---|---|---|---|---|
| | Zeta | Z-average | | | | | | |
| | Potential | diameter | Pk 1 | Pk 2 | Pk 3 | Pk 1 | Pk 2 | Pk 3 |
| | (mV) | (nm) | diameter* | diameter | diameter | Area % | Area % | Area % |
| Heparin only | −0.42 | 177.30 | 216.97 | 1627.33 | 0.00 | 99.40 | 0.60 | 0.00 |
| 0.05% | −1.89 | 175.80 | 202.87 | 3108.33 | 0.00 | 98.40 | 1.60 | 0.00 |
| 0.10% | −1.17 | 175.73 | 198.77 | 3292.00 | 0.00 | 98.97 | 1.03 | 0.00 |
| 0.50% | −3.24 | 172.73 | 70.62 | 2442.66 | 0.00 | 98.24 | 1.76 | 0.00 |
| 1.00% | −5.90 | 168.33 | 200.83 | 1694.48 | 0.00 | 98.90 | 1.10 | 0.00 |
| 5.00% | −11.07 | 175.73 | 182.07 | 3180.00 | 0.00 | 96.63 | 3.37 | 0.00 |
| 10.00% | −14.83 | 166.87 | 180.40 | 3256.00 | 0.00 | 98.67 | 1.33 | 0.00 |

Percentages reflect % contamination of heparin with OSCS, w/w. All numbers are an average of three runs.

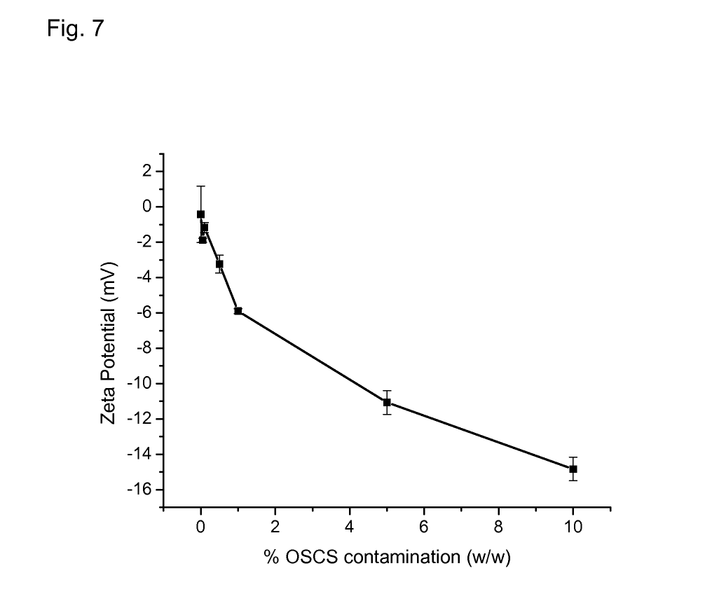

From Table 13, we can see that differences between each contamination level are ambiguous using method ‘I’. Z-average diameters vary little, and the changes between distribution peaks 1 and 2 are very variable. Results obtained are far less ambiguous using method ‘D’ for digestion. We see that addition of OSCS in as low an amount as 0.05% w/w results in a change in liposome aggregate zeta potential of 450%. The smallest change, obtained upon addition of 0.10% OSCS by weight, results in a zeta potential change of 279%. These results are graphed in FIG. 7.

### CONCLUSIONS

The considerable changes in zeta potential between pure heparin and such low OSCS contamination levels lead us to conclude that method ‘D’ is a suitable digestion method for heparin before testing with our liposomal aggregation method. This digestion has increased the sensitivity of our method to at least 0.05% contamination with OSCS, far below the FDA's standard of 0.3%.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for determining whether a composition comprises a charged contaminant, the method comprising:
combining a test composition with lipid particles to form a mixture,
wherein the test composition comprises an analyte,
and either
i) determining the zeta potential of the mixture and comparing it to the zeta potential of a control mixture comprising the lipid particles and a reference composition comprising the analyte of known purity, wherein detection of a significant difference between the zeta potential of the mixture and the zeta potential of the control mixture indicates the presence of the charged contaminant in the test composition, or
ii) determining the average aggregate diameter of liposome aggregates in the mixture and comparing it to the average aggregate diameter of a control mixture comprising the lipid particles and a reference composition comprising the analyte of known purity, wherein detection of a significant difference between the average aggregate diameter of the mixture and the average aggregate diameter of the control mixture indicates the presence of the charged contaminant in the test composition.

2. The method of claim 1 wherein the analyte comprises a polymer.

3. The method of claim 2 wherein the polymer comprises a polynucleotide.

4. The method of claim 3 wherein the polynucleotide comprises a supercoiled DNA, and wherein the charged contaminant comprises a relaxed polynucleotide.

5. The method of claim 4 wherein the lipid particles comprise amphipathic molecules having a positively charged hydrophilic region.

6. (canceled)

7. The method of claim 2 wherein the polymer comprises heparin.

8. The method of claim 7 wherein the charged contaminant comprises glycosaminoglycans (GAGs) that are over-sulfated or under-sulfated.

9. The method of claim 8 wherein the charged contaminant comprises over-sulfated GAGs.

10. The method of claim 9 wherein the over-sulfated GAG is selected from dermatan sulfate, chondroitin sulfate, and the combination thereof.

11. The method of claim 7 wherein the lipid particles comprise amphipathic molecules having a zwitterionic hydrophilic region.

12. The method of claim 11 wherein at least one amphipathic molecule comprises at least one hydrophobic chain that is unsaturated.

13. The method of claim 12 wherein the at least one amphipathic molecule comprising at least one hydrophobic chain that is unsaturated is present in the lipid particle at a concentration of at least 99 mol %.

14-15. (canceled)

16. The method of claim 2 wherein the polymer comprises a polypeptide.

17. The method of claim 1 wherein the analyte comprises an organic molecule.

18. The method of claim 1 wherein the zeta potential of the mixture is decreased by at least 5% compared to the control mixture.

19. The method of claim 1 wherein the average aggregate diameter of liposome aggregates in the mixture is decreased by at least 5% compared to the control mixture.

20-24. (canceled)

25. The method of claim 1 wherein the mixture further comprises a multivalent cation.

26-28. (canceled)

29. A method for enriching an analyte, the method comprising:
combining a test composition with lipid particles and multivalent cations to form a mixture,
wherein the test composition comprises an analyte and a contaminant;
incubating the mixture under conditions suitable for forming a complex comprising the analyte bound to the lipid particle;
separating the complex from the contaminant.

30-31. (canceled)

32. The method of claim 29 wherein the analyte is a polynucleotide.

33-34. (canceled)

35. The method of claim 32 wherein the contaminant comprises a polymer.

36-37. (canceled)
